(19) United States
(12) Patent Application Publication
(10) Pub. No.: US 2026/0120438 A1
(43) Pub. Date: Apr. 30, 2026

(54) TRAINING DATA CREATING DEVICE, INFORMATION SUPPORT DEVICE, ENDOSCOPE SYSTEM, TRAINING DATA CREATING METHOD, AND INFORMATION STORAGE MEDIUM

(71) Applicant: OLYMPUS CORPORATION, Tokyo (JP)

(72) Inventor: Katsumi HIRAKAWA, Yokohama-shi (JP)

(73) Assignee: OLYMPUS CORPORATION, Tokyo (JP)

(21) Appl. No.: 19/317,549

(22) Filed: Sep. 3, 2025

(30) Foreign Application Priority Data
Oct. 25, 2024 (JP) 2024-188034

(51) Int. Cl. G06V 10/774; G06T 7/00; G06V 10/764; G16H 30/40

(52) U.S. Cl. CPC G06V 10/774; G06T 7/0012; G06V 10/764; G16H 30/40; G06T 2207/10068; G06T 2207/30101

(57) ABSTRACT

The present embodiment relates to a training data creating device including one or more processors that perform processing of generating training data for machine learning. The one or more processors plot a plurality of endoscopic images of a training device in the feature space based on individual features, determine classes for the endoscopic images, and set a set region for each of the determined classes. The one or more processor newly generate training data so that the number of pieces of training data newly generated in a first region which is an intersection region of the set regions for each class is greater than the number of pieces of training data newly generated in a second region which is a region excluding the first region from a union region of the set regions for each class.

### CROSS REFERENCE TO RELATED APPLICATIONS

This application is based upon and claims the benefit of priority to Japanese Patent Application No. 2024-188034 filed on October 25, 2024, the entire contents of which are incorporated herein by reference.

### BACKGROUND

In the medical and other fields, techniques for automatically recognizing surgical scenes by machine learning are known. The time required for each surgical scene is different. Thus, if training image data is created based on still images sampled at regular intervals from moving images captured by an imager of an endoscope, the amounts of training for the scenes will not be uniform. Japanese Unexamined Patent Application Publication No. 2022-118654 discloses a technique for additionally generating training data by transforming existing captured image data.

### SUMMARY

In accordance with one of some aspect, there is provided a training data creating device including one or more processors configured to perform processing of generating training data for machine learning. The one or more processors plot a plurality of endoscopic images of a training device in a feature space based on individual features, determine classes for the endoscopic images, set a set region for each of the determined classes, and newly generate the training data so that a number of pieces of the training data newly generated in a first region is greater than a number of pieces of the training data newly generated in a second region, the first region being an intersection region of the set regions for each class, the second region being a region excluding the first region from a union region of the set regions for each class.

In accordance with one of some aspect, there is provided an information support device including: a memory configured to store a trained model trained by machine learning with the training data generated by the training data creating device described above; and one or more processors. The one or more processors output a result of inference from the endoscopic image based on the trained model.

In accordance with one of some aspect, there is provided an endoscope system including: the information support device described above; and an endoscope.

In accordance with one of some aspect, there is provided a training data creating method including: plotting a plurality of endoscopic images of a training device in a feature space based on individual features; determining classes for the endoscopic images; setting a set region for each of the determined classes; and newly generating training data so that a number of pieces of training data newly generated in a first region is greater than a number of pieces of the training data newly generated in a second region, the first region being an intersection region of the set regions for each class, the second region being a region excluding the first region from a union region of the set regions for each class.

In accordance with one of some aspect, there is provided a non-transitory information storage medium that stores a program for causing a computer to execute: plotting a plurality of endoscopic images of a training device in a feature space based on individual features; determining classes for the endoscopic images; setting a set region for each of the determined classes; and newly generating training data so that a number of pieces of the training data newly generated in a first region is greater than a number of pieces of the training data newly generated in a second region, the first region being an intersection region of the set regions for each class, the second region being a region excluding the first region from a union region of the set regions for each class.

### BRIEF DESCRIPTION OF THE DRAWINGS

FIG. 1 is a block diagram illustrating a configuration example of a system including a training data creating device and an endoscope system.

FIG. 2 is a diagram illustrating an example of the endoscope system for use in laparoscopic surgery.

FIG. 3 is a diagram schematically illustrating arteries related to the large intestine.

FIG. 4 is a diagram illustrating input/output data for a trained model.

FIG. 5 is a table illustrating an example of a process chart for sigmoidectomy.

FIG. 6 is a flowchart illustrating an example of a treatment flow to which a technique of the present embodiment is applied.

FIG. 7 is an example of a predetermined table.

FIG. 8 is a diagram illustrating the acquisition timing for endoscopic images.

FIG. 9 is a flowchart illustrating an example of processing pertaining to the technique of the present embodiment.

FIG. 10 is a chart illustrating an example of a feature space.

FIG. 11 is a chart illustrating a set region of a first class and a set region of a second class.

FIG. 12 is a chart illustrating a first region and a second region.

FIG. 13 is a flowchart illustrating an example of processing pertaining to creation of training data.

FIG. 14 is a flowchart illustrating an example of processing pertaining to creation of an interpolated image.

FIG. 15A is a diagram illustrating a technique for determining that a selected instance is located in the second region.

FIG. 15B is a diagram illustrating a technique for determining that a selected instance is located in the first region.

FIG. 16 is a flowchart illustrating an example of processing pertaining to generation of an interpolated image based on a third instance.

FIG. 17 is a diagram illustrating the third instance, a fourth instance, and a fifth instance.

FIG. 18 is a diagram illustrating a method for determining the time pertaining to the third instance.

FIG. 19 is a diagram illustrating another configuration example of the training data creating device.

FIG. 20 is a flowchart illustrating another example of processing pertaining to creation of an interpolated image.

FIG. 21 is a diagram illustrating generation of an extended image.

FIG. 22 is a diagram illustrating another example of the technique of the present embodiment.

FIG. 23 is a diagram illustrating another example of the technique of the present embodiment.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being "connected" or "coupled" to a second element, such description includes embodiments in which the first and second elements are directly connected or coupled to each other, and also includes embodiments in which the first and second elements are indirectly connected or coupled to each other with one or more other intervening elements in between.

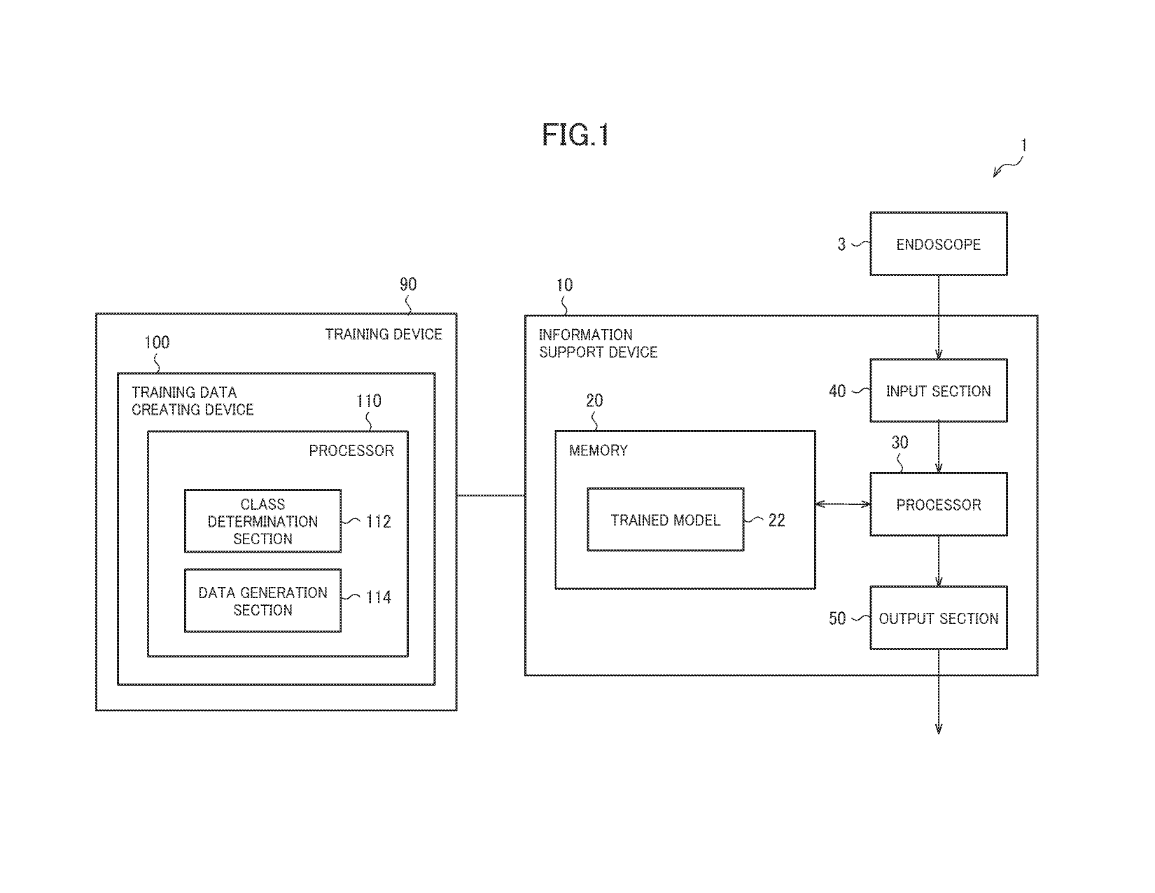

FIG. 1 is a block diagram illustrating a configuration example of a system including an endoscope system 1 and a training device 90 in the present embodiment. FIG. 1 is a block diagram illustrating a configuration example of the endoscope system 1 including a training data creating device 100. In FIG. 1, the training device 90 includes the training data creating device 100. The training data creating device 100 includes a processor 110.

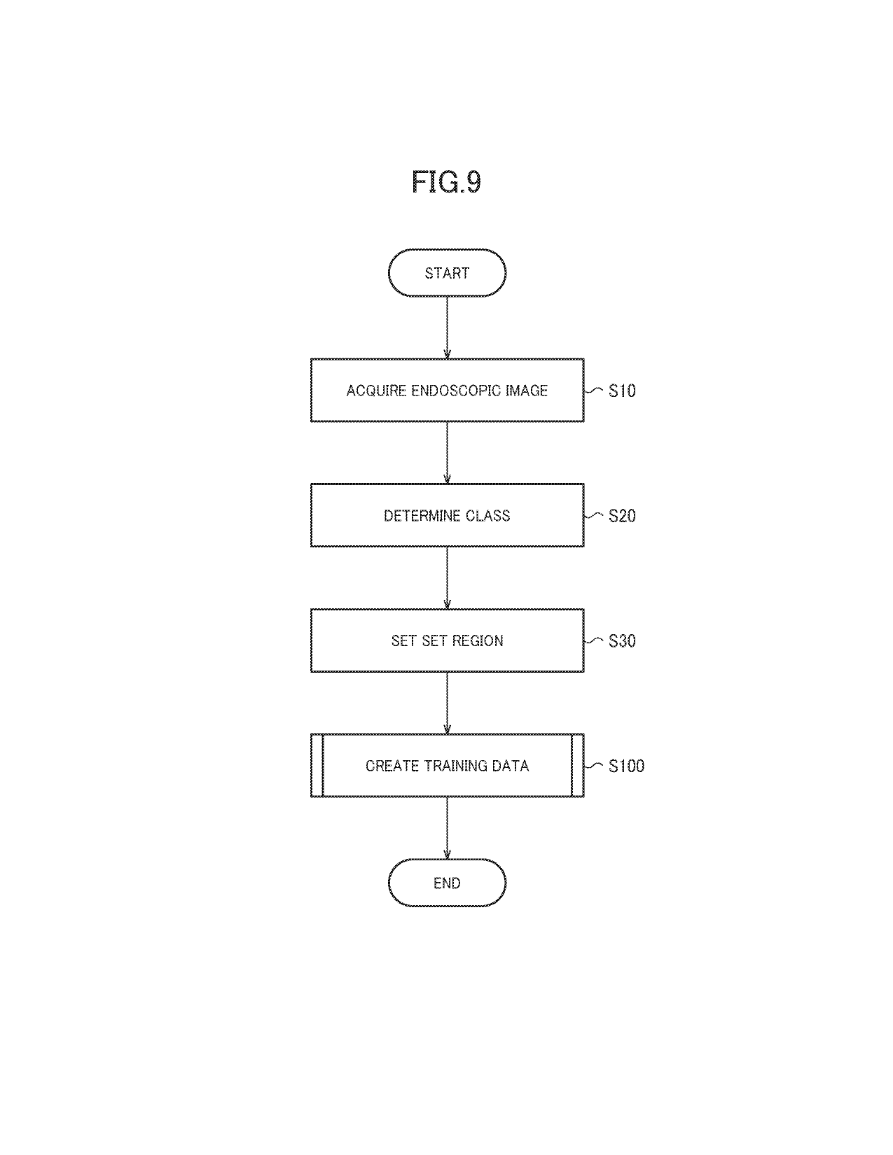

The processor 110 of the training data creating device 100 according to the present embodiment (hereinafter referred to simply as "processor 110") is configured with the following hardware. The hardware can include at least one of a circuit that processes digital signals and a circuit that processes analog signals. For example, the hardware can be configured with one or more circuit devices or one or more circuit elements mounted on a circuit board. One or more circuit devices are, for example, ICs. One or more circuit elements are, for example, capacitors. For example, the training data creating device 100 in the present embodiment may include a memory not illustrated in FIG. 1 and the processor 110 that operates based on information stored in the memory. This configuration allows the processor 110 to function as a class determination section 112, a data generation section 114, and the like. As will be described later in FIG. 9 and the subsequent drawings, the main entity for processing and the like pertaining to a technique of the present embodiment is consistently the processor 110 for convenience of explanation. The information stored in the memory is, for example, a program and various data. A central processing unit (CPU), a graphics processing unit (GPU), a digital signal processor (DSP), or the like can be used as the processor 110. The memory may be a volatile memory such as a static random access memory (SRAM) or a dynamic random access memory (DRAM), a nonvolatile memory such as a read only memory (ROM), a magnetic storage device such as a hard disk device, or an optical storage device such as an optical disk device. For example, the memory stores computer-readable instructions, and the instructions are executed by the processor 110 to cause the function of each section to be implemented as processing. As used herein, the instructions may be instructions of an instruction set that constitutes a program or may be instructions to instruct the hardware circuit of the processor 110 to operate.

The program, which is not illustrated in the drawing, can be stored in a non-transitory information storage medium which is a medium that can be read by a computer, for example. The information storage medium can be implemented by, for example, an optical disk, a memory card, a hard disk device, a nonvolatile memory, or the like.

The training device 90 further includes a not-illustrated processor (hereinafter

referred to as "training device processor" for the sake of convenience) and a memory, in addition to the training data creating device 100. The memory of the training device 90 stores a machine learning program and training data, and the training device processor functions as a machine learning section to generate or update a trained model 22 described later.

In FIG. 1, the training device 90 includes the training data creating device 100, but the training device 90 may further function as the training data creating device 100. In this case, the memory of the training device 90 may further include a program pertaining to processing performed by the training data creating device 100, and the training device processor may function as a machine learning section and a training data generation section.

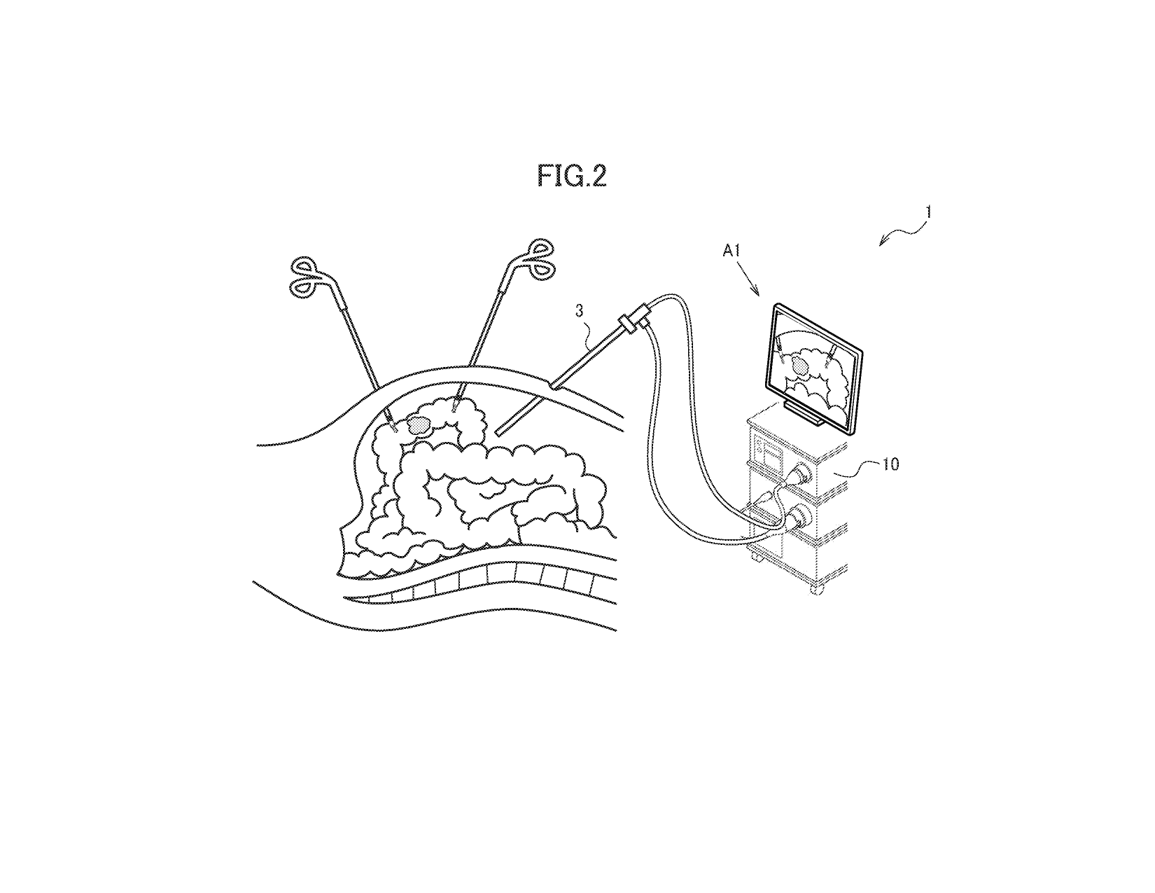

The trained model 22 generated by the training device 90 configured in this way is stored in a memory 20 of an information support device 10 through data transmission/reception and the like via a not-illustrated communication interface. The example of the system illustrated in FIG. 1 is a configuration example in a learning phase. When inference is performed based on the trained model 22, the training device 90 may be separated from the system illustrated in FIG. 1, and only the endoscope system 1 may be used and applied in actual endoscopic surgery or the like as illustrated in FIG. 2.

The endoscope system 1 in the present embodiment includes an endoscope 3 and the information support device 10. The information support device 10 includes the memory 20 and a processor 30. The memory 20 stores the trained model 22. The processor 30 of the information support device 10 (hereinafter referred to simply as "processor 30") can be configured with hardware similar to that of the processor 110 included in the training data creating device 100 described above. The information support device 10 includes the memory 20 and the processor 30, whereby the processor 30 functions as an inference section. In other words, the memory 20 stores a program and the like to allow the processor 30 to function as an inference section. The memory 20 is a non-transitory information storage medium that can be implemented by a disk, a memory card, a hard disk device, a nonvolatile memory, or the like.

As illustrated in FIG. 1, the information support device 10 in the present embodiment may further include an input section 40 and an output section 50. Although one input section 40 and one output section 50 are illustrated in FIG. 1, a plurality of input sections 40 and a plurality of output sections 50 may be provided. In the present embodiment, for convenience of explanation, hardware that functions as an input interface described later is generally referred to as the input section 40, and hardware that functions as an output interface is generally referred to as the output section 50.

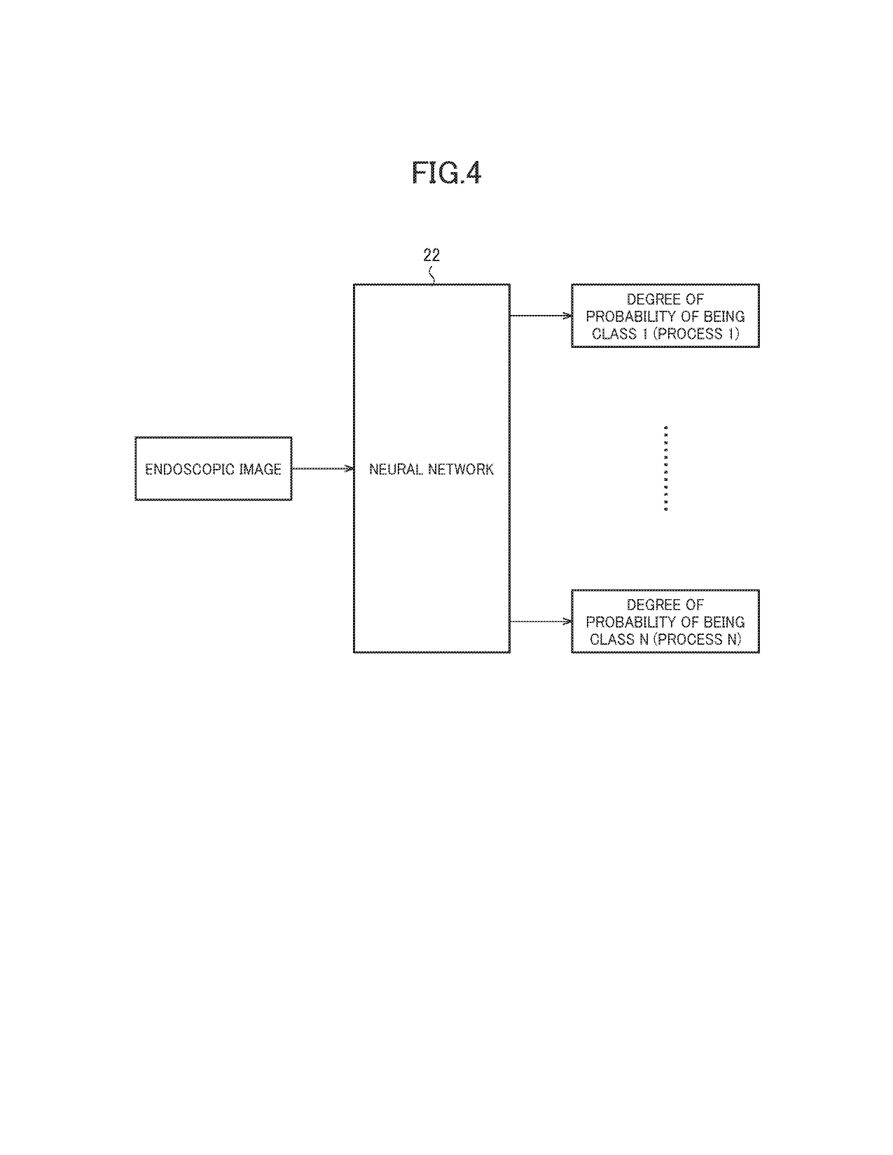

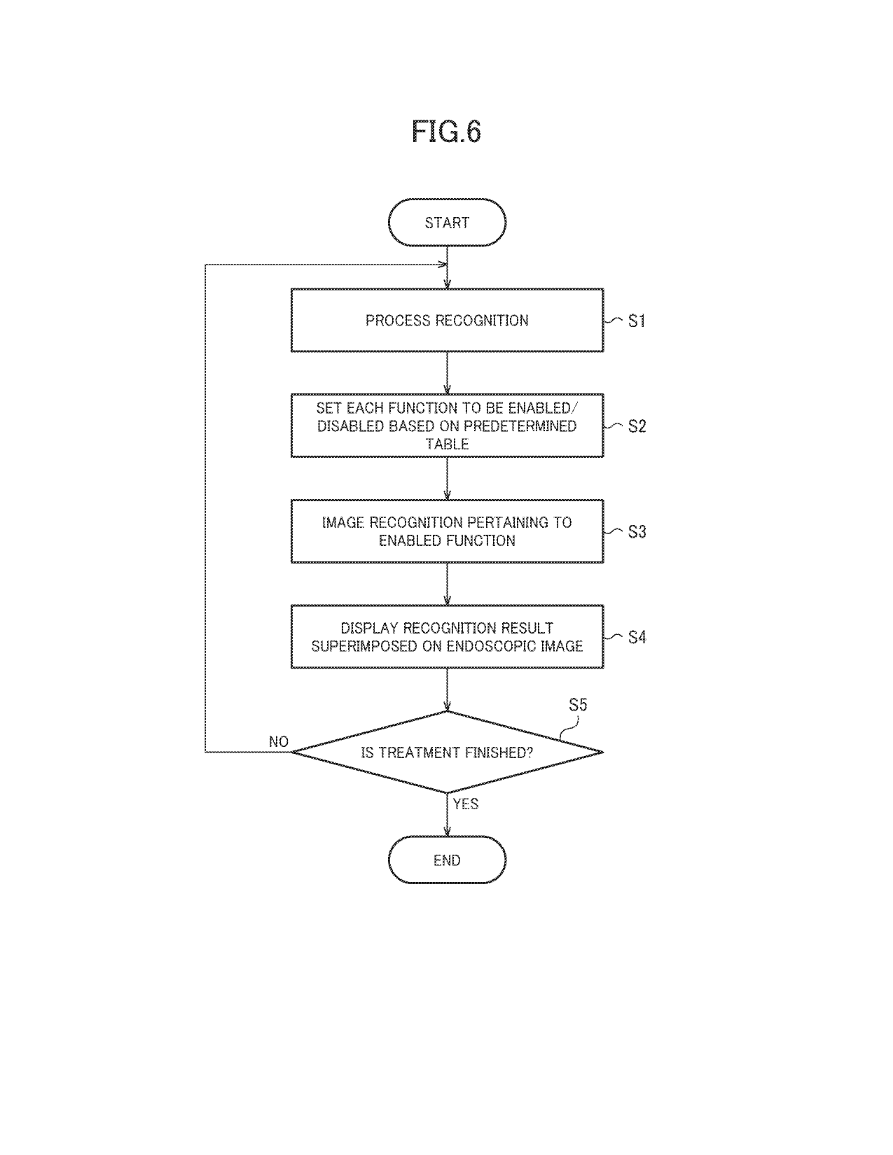

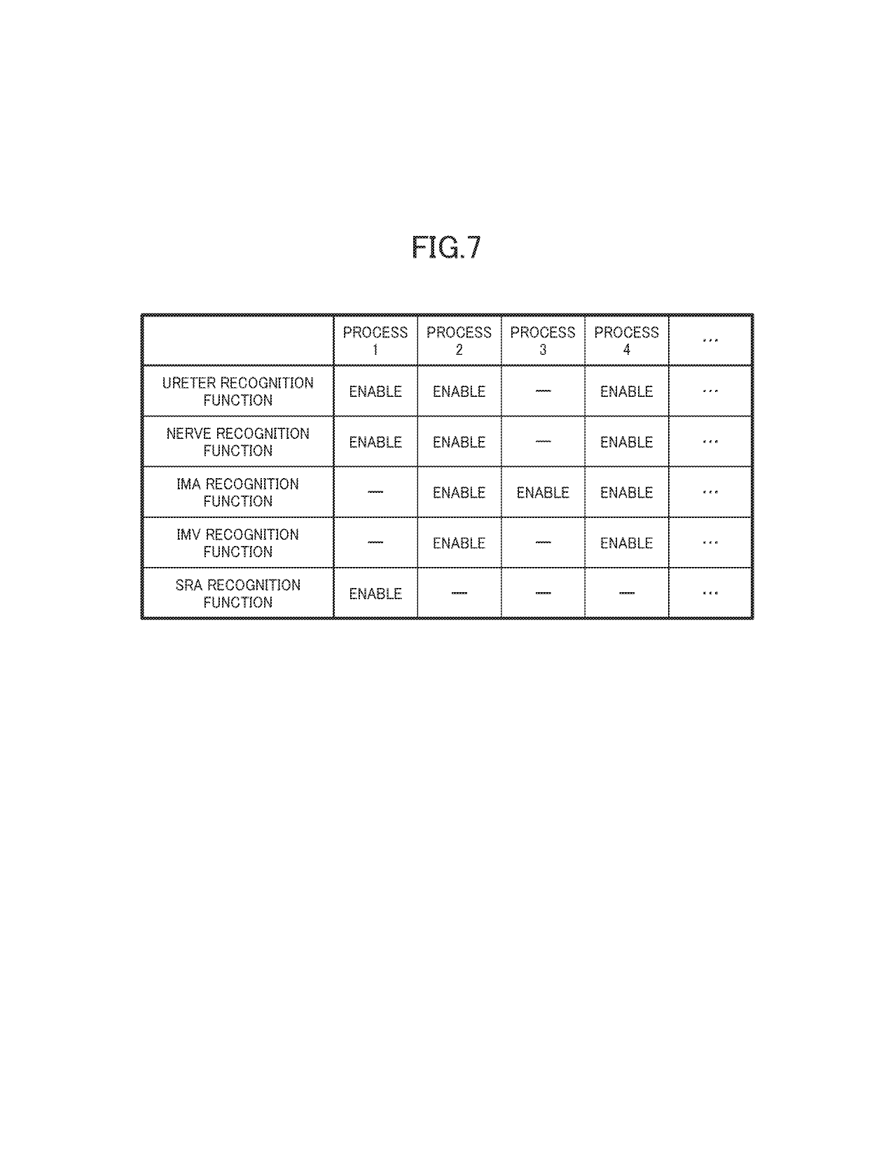

Although FIG. 1 illustrates one trained model 22 stored in the memory 20, the information support device 10 in the present embodiment may include multiple types of trained models, which will be detailed later with reference to FIG. 6 and FIG. 7. In other words, the processor 30 in the present embodiment may be able to perform processing using a plurality of trained models simultaneously. In the present embodiment, for convenience of explanation, the trained model pertaining to the processing in step S1 in FIG. 6 described later, which is trained by machine learning described later with reference to FIG. 4, is accompanied by a reference sign and referred to as "trained model 22".

The endoscope 3 in the present embodiment is, for example, a rigid endoscope having an insertion section most of which is rigid. The rigid endoscope is well known and therefore its configuration is not illustrated in detail. A configuration that includes an imager at a distal end of the endoscope 3 is widely used. In this way, the input section 40 of the information support device 10 can receive an image signal from the imager via a not-illustrated cable and establish a relationship whereby the processor 30 generates a display image based on the received image signal. In this configuration, the information support device 10 is connected to the endoscope 3, and the information support device 10 is connected to a display denoted by A1 in FIG. 2, so that the endoscope system 1 can be constructed in which the display image captured by the imager is displayed on the display via the output section 50. This allows a user to perform treatment on living tissue with a treatment tool while observing the living tissue in the body cavity in endoscopic surgery as illustrated in FIG. 2. The user in the present embodiment refers to, for example, a surgeon who handles a treatment tool, a scopist who operates the endoscope 3, and all other persons involved in treatment.

In the present embodiment, still images acquired by the imager at regular time intervals in units of first times in each case are referred to as "endoscopic images". A set of still images captured by the imager is referred to as "endoscopic video" for the sake of convenience. In other words, the processor 30 in the present embodiment acquires time-series endoscopic images in manipulation using the endoscope 3. Specifically, for example, in the present embodiment, when a video signal of an endoscopic image is input to the processor 30 from the imager via the input section 40, the processor 30 performs first processing of generating an endoscopic image based on the received video signal and outputting the endoscopic image via the output section 50, and second processing of performing inference based on the trained model 22 using the generated endoscopic image as input data. In other words, although not specifically illustrated in the drawing, the information support device 10 includes an input interface as hardware for inputting endoscopic image data to the trained model 22 and an output interface for outputting output data based on an inference result. This allows the user to smoothly perform treatment while observing the endoscopic image, as described later. The second processing here corresponds to process recognition (step S1) described later with reference to FIG. 6.

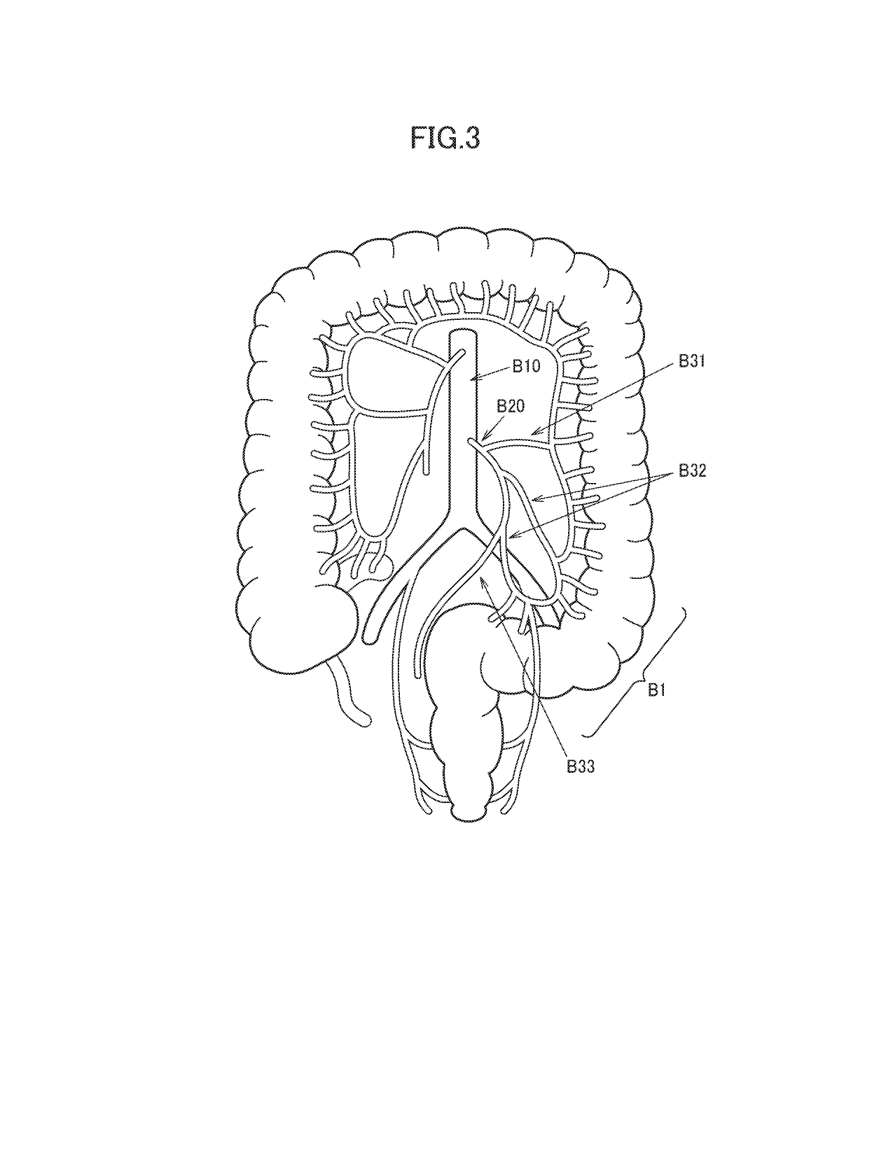

One of examples of manipulation to which the technique of the present embodiment can be applied is sigmoidectomy. The sigmoidectomy is treatment that aims for radical cure by resecting a sigmoid colon when a lesion such as cancer occurs in the sigmoid colon denoted by B1 in a large intestine schematically illustrated in FIG. 3. In sigmoidectomy, it is necessary to consider the handling of not only the sigmoid colon but also arteries, veins, nerves, and other tissues related to sigmoidectomy. In the case of arteries, for example, the related arteries include abdominal aorta denoted by B10 in FIG. 3, inferior mesenteric artery denoted by B20 (hereafter referred to as IMA), left colic artery denoted by B31 (hereafter referred to as LCA), sigmoid artery denoted by B32, superior rectal artery denoted by B33 (hereinafter referred to as SRA), and the like.

Although not illustrated in the drawing, the related veins include inferior mesenteric vein (hereafter referred to as IMV) and the like. How these tissues are handled is determined as appropriate depending on a case. For example, when the lesion is cancer, the extent of lymph nodes to be resected together with the sigmoid colon is determined according to the progress of the cancer, and predetermined lymph node resection and vascular treatment are performed.

The endoscope 3 in the present embodiment is not limited to the rigid endoscope described above, but may be any other type of endoscope, such as a soft endoscope. In other words, the technique of the present embodiment can be widely applied to manipulation using the endoscope 3. For example, in the following, a process and the like related to sigmoidectomy using the rigid endoscope will be described. However, the manipulation to which the technique of the present embodiment can be applied is not limited to sigmoidectomy, and the technique of the present embodiment can be widely applied to manipulation using the endoscope 3.

Machine learning in the present embodiment is, for example, supervised learning, and the trained model 22 is generated by supervised learning based on a data set in which input data is associated with a ground truth label. A neural network is included in at least a part of the trained model 22 in the present embodiment. The neural network, which is not illustrated in detail in the drawings, includes an input layer that receives data, an intermediate layer that performs computation based on an output from the input layer, and an output layer that outputs data based on an output from the intermediate layer. The number of intermediate layers is not limited. The number of nodes in each layer in the intermediate layer is not limited. The nodes included in a given layer in the intermediate layer are connected to nodes in an adjacent layer. A weighting factor is set for each connection. Each node multiplies an output from a node in the preceding stage by the weighting factor and obtains a sum of multiplication results. In addition, each node adds a bias to the sum and applies an activation function to the addition result to obtain an output of the node. This processing is successively executed from the input layer toward the output layer to obtain an output of the neural network.Various functions such as sigmoid and ReLU functions are known as the activation function and can be widely applied in the present embodiment.

Training in the neural network is processing of determining an appropriate weighting factor. The weighting factor here includes a bias. In the example illustrated in FIG. 1, the training device processor functions as the machine learning section and performs processing of generating or updating the trained model 22. The processor 30 inputs input data of training data to the neural network and obtains an output by performing forward computation using the weighting factor at that moment. The processor 30 computes an error function based on the output and the ground truth label of the training data. The weighting factor is then updated so that the error function is reduced. In the updating of the weighting factor, for example, back propagation can be used, which updates the weighting factor from the output layer toward the input layer.

Models with various configurations are known for the neural network and can be widely applied in the present embodiment. For example, the neural network may be a convolutional neural network (CNN), a recurrent neural network (RNN), or other models. When CNN or the like is used, input data of training data is input to a model, and an output is obtained by performing forward computation in accordance with a model configuration using the weighting factor at that moment. The error function is calculated based on the output and the ground truth label, and the weighting factor is updated so that the error function is reduced. For example, backward propagation can also be used when the weighting factor of the CNN or the like is updated.

In a single endoscopic surgery by a given user, time-series endoscopic image data is acquired. For example, the processor 30 uses data extracted from the time-series endoscopic image data as an input to the neural network.

The output of the neural network is, for example, information representing a process stage when the processes in endoscopic surgery are classified into N stages. N is an integer equal to or greater than 2. For example, the output layer of the neural network has N nodes. A first node is information indicating a degree of probability that the process corresponding to the input endoscopic image belongs to class 1. This is applicable to a second node to an Nth node, and these nodes are information representing the degrees of probability that input data belongs to class 2 to class N, respectively. For example, when the output layer is a known softmax layer, N outputs are a set of probability data of which sum is 1. Class 1 to class N are classes corresponding to process 1 to process N, respectively.

In this way, the trained model 22 in the present embodiment is generated by supervised learning based on a data set in which input data including an endoscopic image is associated with a ground truth label which is a process stage. The technique of the present embodiment does not preclude the application of other learning methods such as semi-supervised learning and self-supervised learning.

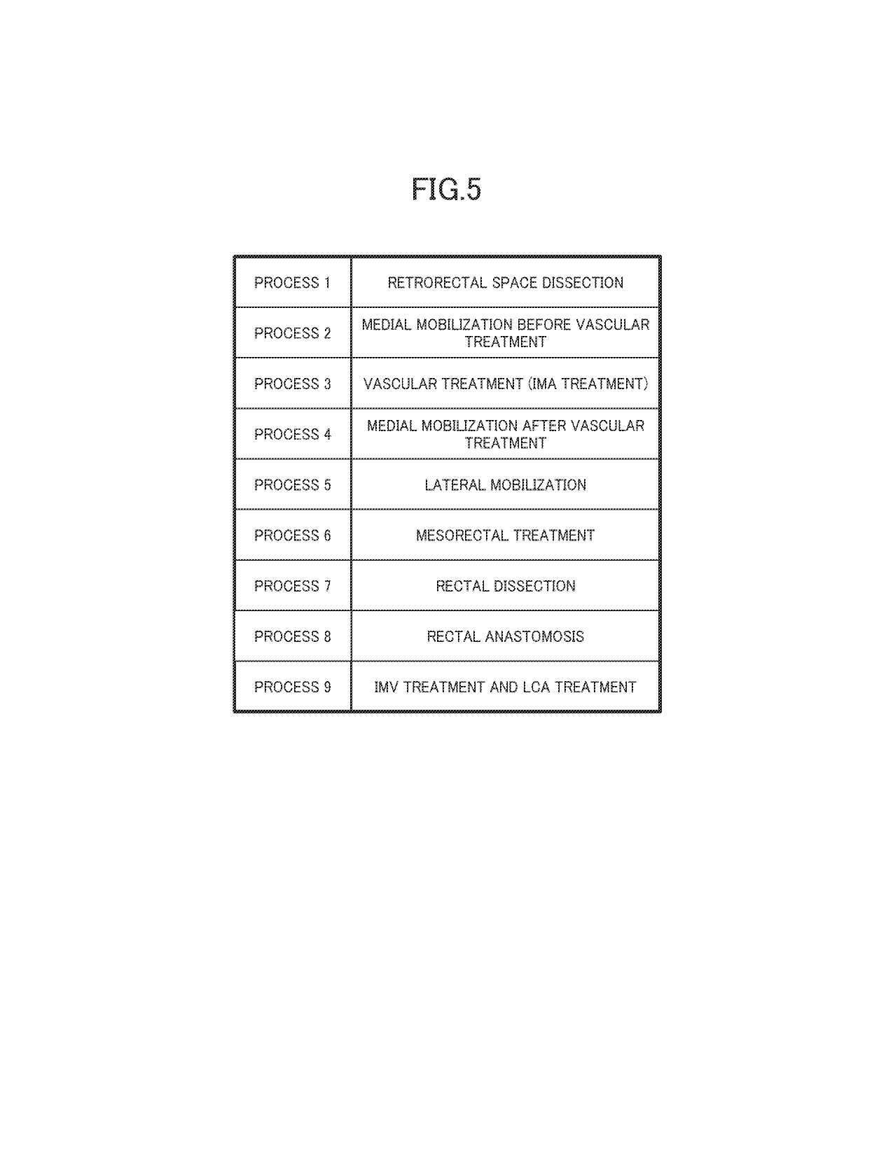

For example, in the sigmoidectomy described above, processes are classified in stages, for example, as illustrated in FIG. 5. In FIG. 5, "process 1" is "retrorectal space dissection", "process 2" is "medial mobilization before vascular treatment", "process 3" is "vascular treatment", "process 4" is "medial mobilization after vascular treatment", "process 5" is "lateral mobilization", "process 6" is "mesorectal treatment", "process 7" is "rectal dissection", "process 8" is "rectal anastomosis", and "process 9" is "IMV treatment and LCA treatment". The process stages are illustrated in FIG. 5 by way of example and are not limited to these. The parenthesized "IMA treatment" in "process 3" indicates an example of "vascular treatment", and which vessels to treat in process 3 is determined by a surgery procedure.

The trained model 22 trained by machine learning in this way can be applied to treatment including, for example, the processing illustrated in the flowchart in FIG. 6. In FIG. 6, the processor 30 performs process recognition (step S1) and then sets a function described later to be enabled or disabled based on a predetermined table (step S2). The processor 30 then performs image recognition (step S3) pertaining to the enabled function, displays the recognition result superimposed on an endoscopic image (step S4), and then determines whether the treatment has been finished (step S5). If it is determined that the treatment has not been finished (NO in step S5), the processor 30 performs step S1 again. If it is determined that the treatment been finished (YES in step S5), the flow ends.

In step S1, the processor 30 reads the trained model 22 and infers a process stage based on the endoscopic image input from the endoscope 3. In other words, step S1 is processing in which the processor 30 functions as the inference section as described above with reference to FIG. 4.

In step S2, the processor 30 enables a function necessary for the process pertaining to a class inferred in step S1. Specifically, for example, the predetermined table illustrated in FIG. 7 is stored in the memory 20. FIG. 7 is an example of a part of the predetermined table in a case where the treatment is sigmoidectomy. For example, if the processor 30 estimates class 1 in step S1, the processor 30 sets a ureter recognition function, a nerve recognition function, and an SRA recognition function to be enabled in step S2. The ureter recognition function here refers to a function that performs image processing on the endoscopic image appearing on the display so that the user can recognize a region where the ureter is located. The region where the ureter is located refers to a region where the ureter can be visually recognized directly from the endoscopic image as well as a region where the ureter is located when the ureter is assumed to be present on the far side of a tissue surface displayed in the endoscopic image. This is applicable to the nerve recognition function, the IMA recognition function, the IMV recognition function, and the SRA recognition function in FIG. 7.

The image processing in the ureter recognition function may use a trained model trained by machine learning, for example, including the CNN described above. In other words, a trained model trained by machine learning with a data set in which the endoscopic image is input data and the region where the ureter is located is output data may be stored in the memory 20. Thus, the processor 30 reads the trained model trained by machine learning on the ureter recognition function and performs, for example, processing such as semantic segmentation on the endoscopic image in step S3. Similarly, the processor 30 reads the trained model trained by machine learning on the nerve recognition function and the trained model trained by machine learning on the SRA recognition function, and performs processing such as semantic segmentation on the endoscopic image in step S3.

The processor 30 then superimposes and displays marker information segmented for each of the ureter, nerve, and SRA on the endoscopic image in step S4. Step S5 is NO until the treatment related to process 1 is completed. Hence, steps S1 to S4 are repeatedly performed each time an endoscopic image is acquired.

The user then completes the treatment pertaining to process 1 and begins the treatment pertaining to process 2. Since this changes the endoscopic image captured, the processor 30 estimates class 2 from the endoscopic image acquired in step S1. In the table shown in FIG. 5, an interval may be further provided between each process, and the processor 30 may infer from the endoscopic image that it is during a period pertaining to the interval. The processor 30 may set the functions illustrated in FIG. 6 to be disabled during the period pertaining to the interval.

If process 2 is inferred from the endoscopic image in step S1, the processor 30 sets the ureter recognition function, the nerve recognition function, the IMA recognition function, and the IMV recognition function to be enabled in step S2. In other words, the trained model pertaining to the ureter recognition function, the trained model pertaining to the neural recognition function, the trained model pertaining to the IMA recognition function, and the trained model pertaining to the IMV recognition function are read. The processor 30 then performs processing such as semantic segmentation on the endoscopic image for the sites pertaining to ureter, nerve, IMA, and IMV in step S3, and displays marker information for the ureter, nerve, IMA, and IMV superimposed on the endoscopic image in step S4. Similar processing is performed for process 3 and subsequent processes. FIG. 7 indicates that only the IMA recognition function is set to be enabled when "IMA processing" is performed as an example of "vascular treatment" as described above with reference to FIG. 6.

In sigmoidectomy, it is desirable that the positions of ureter, nerve, IMA, IMV, SRA, and the like can be grasped from the endoscopic image. However, if all of these tissues are segmented in all the processes, the user will not be able to perform the treatment smoothly. Therefore, it is desirable that the segmented tissues are switched

according to the process stage. However, if the user performs the setting of image processing at each process stage, the treatment efficiency is reduced. In this regard, by using the information support device 10 in the present embodiment, the user can perform the treatment efficiently because the tissues subjected to image processing on the endoscopic image are automatically switched when the process stages are switched. In order to improve the inference accuracy of the process stages in such endoscopic surgery, it is desirable to be able to acquire a large number of endoscopic images serving as training data in each process.

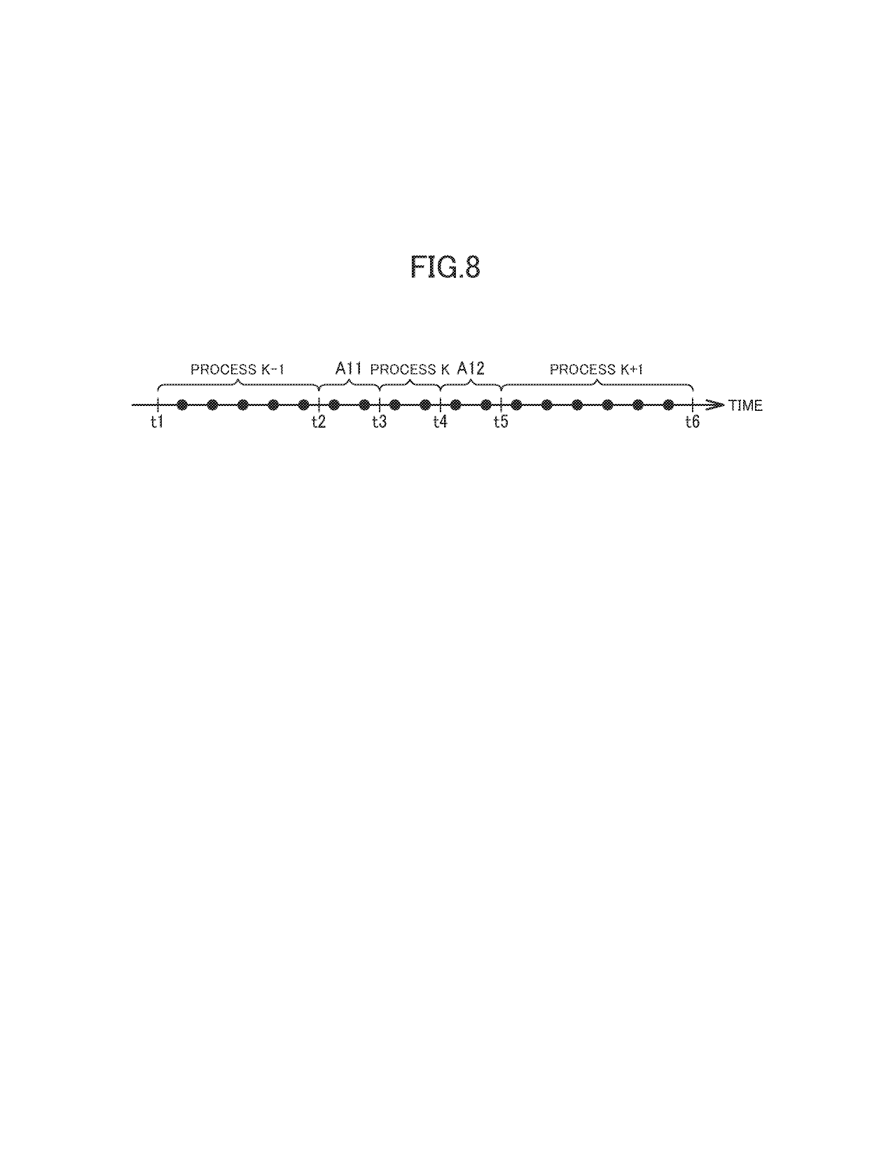

However, the time required for each process is not uniform or the same. For example, as illustrated in FIG. 8, suppose that, in a certain endoscopic surgery, process (K-1) is performed from timing t1 to timing t2, process K is performed from timing t3 to timing t4, and process (K+1) is performed from timing t5 to timing t6. K is a natural number equal to or greater than 2. The number of endoscopic images that can be acquired as training data is proportional to the time required for the process, because endoscopic images are acquired every first time, that is, at regular time intervals, as described above. In FIG. 8, for example, the ratio of the number of endoscopic images as acquired training data is process (K-1):process K:process (K+1) = 5:2:6. Therefore, to improve the accuracy of learning, for example, it is necessary to acquire more training data for process K. The present embodiment relates to a technique for generating training data that leads to improvement in learning accuracy for a process such as process K in which less training data is acquired than in other processes.

For example, when one endoscopic surgery is performed, time-series endoscopic image data (endoscopic video) related to the endoscopic surgery is stored in a not-illustrated memory included in the training device 90, for example, in a directory-style file management structure having a hierarchical structure based on processes. The processor 110 then newly creates training data for a plurality of endoscopic images stored in the memory as training data, using a technique described below. The training device processor then updates the directory to add the newly created training data. Alternatively, a text-format management file may be created based on an image file name

of each endoscopic image stored in the not-illustrated memory included in the training device 90, and the user may manage the management file. The training device processor may then update the management file to add text based on the image file name of the newly created training data. The user may further manage the acquired endoscopic image in association with time information at which the acquired endoscopic image is acquired. Specifically, for example, the endoscopic image data may be in a directory-style file management structure based on processes and time information, or a management file may be created that associates a text name based on the image file name of an endoscopic image with a text name based on time information.

In the following, a process with less training data will be described by way of example. However, the technique described below may be applied to, for example, creation of more training data pertaining to an interval period. The periods denoted by A11 and A12 in FIG. 8 correspond to the interval periods. The period denoted by A11 is a period from timing t2, which is timing when process (K-1) ends, to timing t3, which is timing when process K is started. Similarly, the period denoted by A12 is a period from timing t4, which is timing when process K ends, to timing t5, which is timing when process (K+1) is started.

In the following, a technique that newly creates training data in a process with relatively less training data originally acquired will mainly be described. However, the technique of the present embodiment may be applied for newly creating training data in a process with relatively much training data originally acquired, unless otherwise specified.

Referring to the flowchart in FIG. 9, an example of processing related to the technique of the present embodiment will be described. The processor 110 acquires an endoscopic image (step S10) and then functions as the class determination section 112 to perform class determination (step S20). The processor 110 then functions as the data generation section 114 to set a set region (step S30), and then generates training data (step S100). Step S100 will be detailed later.

In step S10, more specifically, for example, the processor 110 acquires time-

series endoscopic images for a predetermined case from the aforementioned database. The data acquired in step S10 includes time information at which the endoscopic image is acquired, information on the process stage based on the time information, and the like.

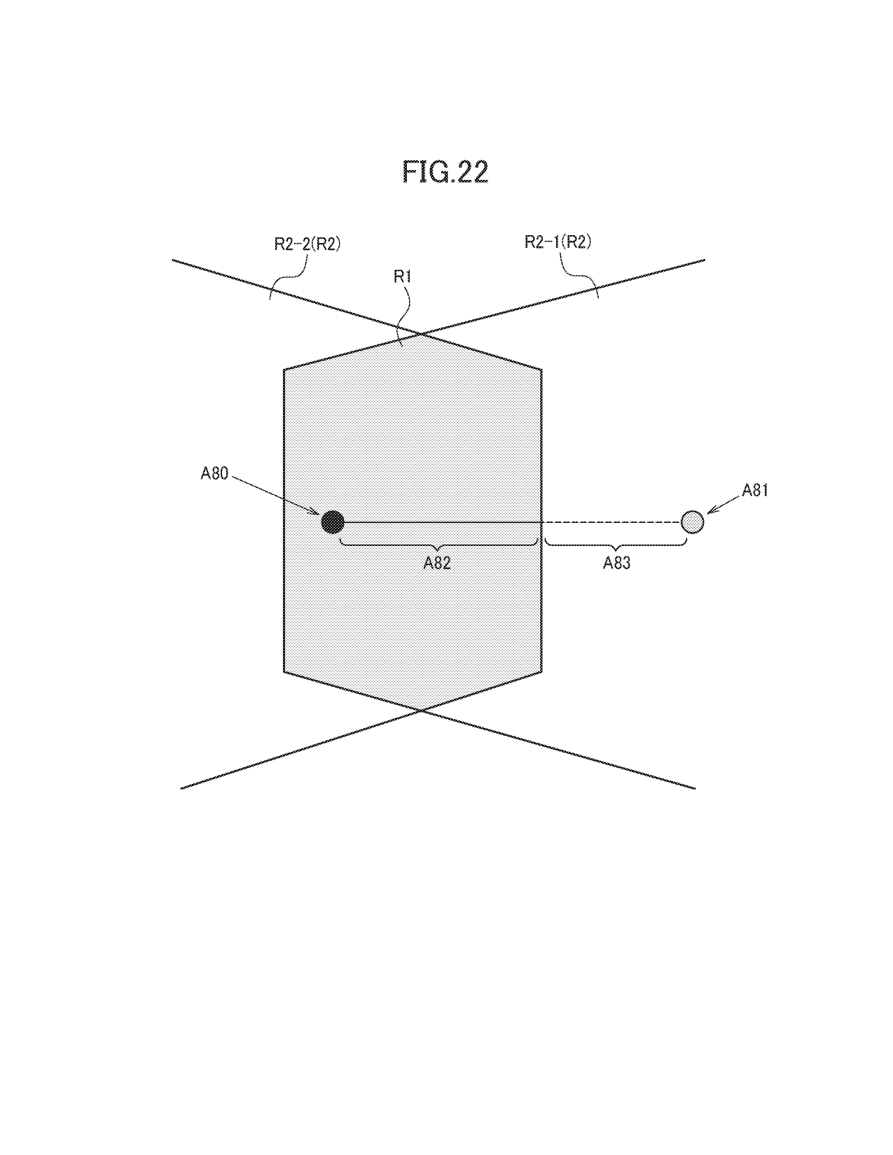

In step S20, for example, the processor 110 determines a class pertaining to a plurality of acquired endoscopic images based on the acquired time information, process information, similarity of images, and the like. In the following, for ease of understanding of the technique of the present embodiment, an example in which a first class is a class with a relatively large number of endoscopic images acquired as training data, a second class is a class with a relatively small number of endoscopic images acquired as training data, and the first class and the second class are used will be described. In the following, it is assumed that the first class and the second class have such a relationship that they are classified by the difference in the number of endoscopic images acquired as training data, and there are no restrictions on other relationships unless otherwise specified. Other relationships are, for example, the relationship between a process pertaining to the first class and a process pertaining to the second class, details of which will be described later with reference to FIG. 22.

The processor 110 then sets a set region (step S30). For example, the processor 110 extracts a feature from the endoscopic image classified in a class in step S20, plots the extracted feature in a feature space, and associates the feature with the determined class. The feature here is also referred to as a feature vector. Examples of the feature include, but are not limited to, edges and gradients of endoscopic images, or their statistics. The feature can be extracted, for example, using a technique such as Scale Invariant Feature Transform (SIFT), Speeded-Up Robust Features (SURF), or Histograms of Oriented Gradients (HOG). However, the feature may be extracted from the endoscopic image by a technique using machine learning. The technique using machine learning here is specifically CNN, but may be a further developed technique of CNN, such as VGG or ResNET.

In the following, for ease of understanding of the technique of the present embodiment, the feature is assumed to be a two-dimensional quantity consisting of

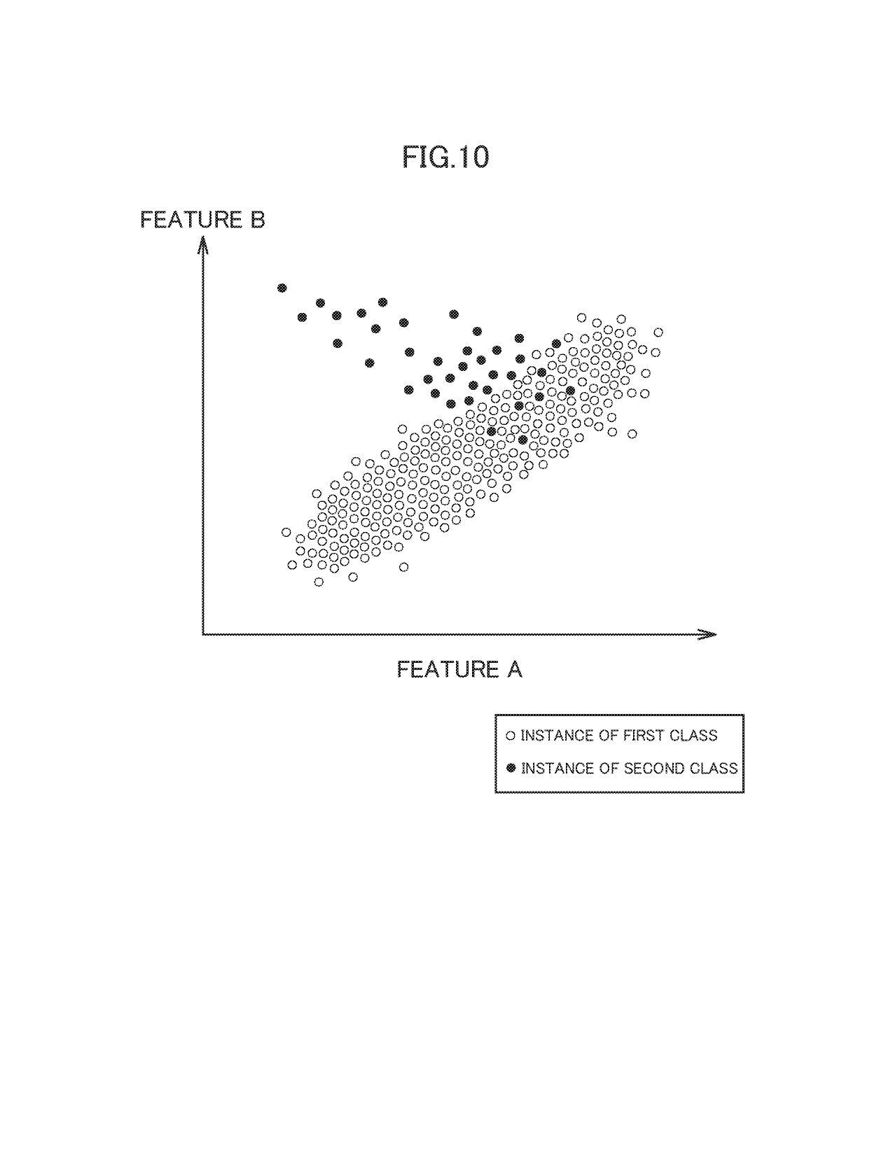

features A and B. However, the technique of the present embodiment is not limited to this and the dimensions of the feature can be extended to three or more. Alternatively, the technique of the present embodiment may include processing that reduces a multidimensional feature space to a lower-dimensional space, such as two or three dimensions, using a technique such as Principal Component Analysis (PCA), so that the user can easily understand the relationship between classes. Specifically, for example, in the feature space illustrated in FIG. 10, instances pertaining to the endoscopic images classified into the first class (hereafter referred to as "instances belonging to the first class") are represented by white circles. Similarly, in the feature space illustrated in FIG. 10, instances pertaining to the endoscopic images classified into the second class (hereafter simply referred to as "instances belonging to the second class") are represented by black circles.

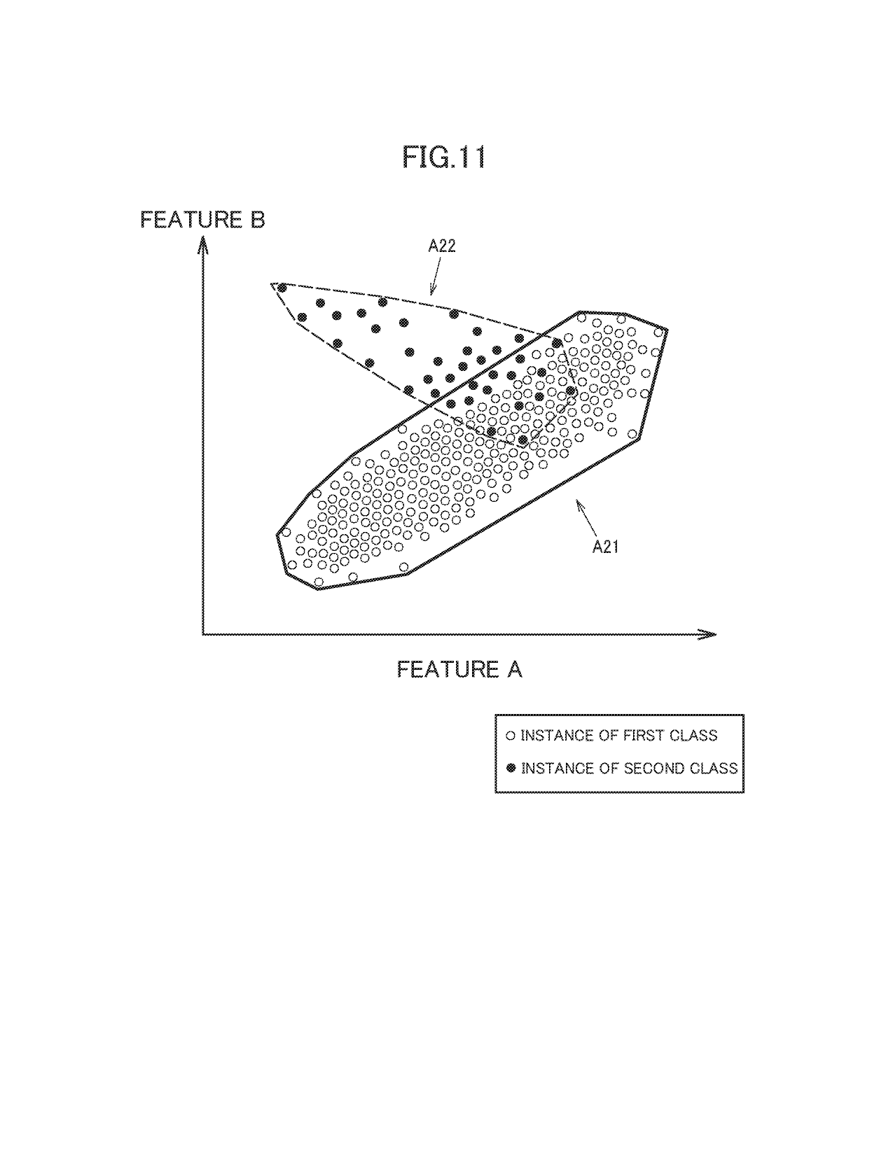

In step S30 in FIG. 9, the processor 110 sets a set region based on a set of instances associated with each class. The shape of the set region is not limited, but in the present embodiment, the processor 110 sets a convex polygonal set region that includes a set of instances belonging to the first class, as denoted by A21 in FIG. 11. Similarly, the processor 110 sets a convex polygonal set region that includes a set of instances belonging to the second class, as denoted by A22 in FIG. 11. In other words, step S30 in the present embodiment includes processing of obtaining a convex hull for each of the set of instances belonging to the first class and the set of instances belonging to the second class. The technique using convex hulls is not limited, and a wide range of known convex hull formation algorithms can be employed.

The processor 110 may set the set region, for example, in further consideration of a distance of the endoscopic image in the feature space in step S30 in FIG. 9. The distance here is specifically, for example, the distance from the centroid of the plot positions of endoscopic images in the feature space to the plot position of each of the endoscopic images. For example, by using a technique similar to a k-means method, the centroid of the plot positions of the endoscopic images in the feature space can be obtained. The k-means method is well known and a detailed explanation is omitted.

The distance from the centroid of the plot positions of the endoscopic images in the feature space to the plot position of each of the endoscopic images is referred to as the first distance for convenience. The first distance may vary by the number of plotted endoscopic images.

For example, the processor 110 may set the set region by further setting a second distance in step S30 in FIG. 9. The second distance is, for example, a fixed distance in the feature space that is set for each class. For example, the processor 110 may compare the first distance with the second distance for each instance belonging to the same class, and if the first distance is shorter than the second distance for all the instances, the processor 110 may set the set region for all the instances. For example, if there is an instance with the first distance longer than the second distance, the processor 110 may exclude such an instance and set the set region for the instances that have not been excluded. In other words, the second distance is a distance serving as a reference by which belonging to the set region is accepted.

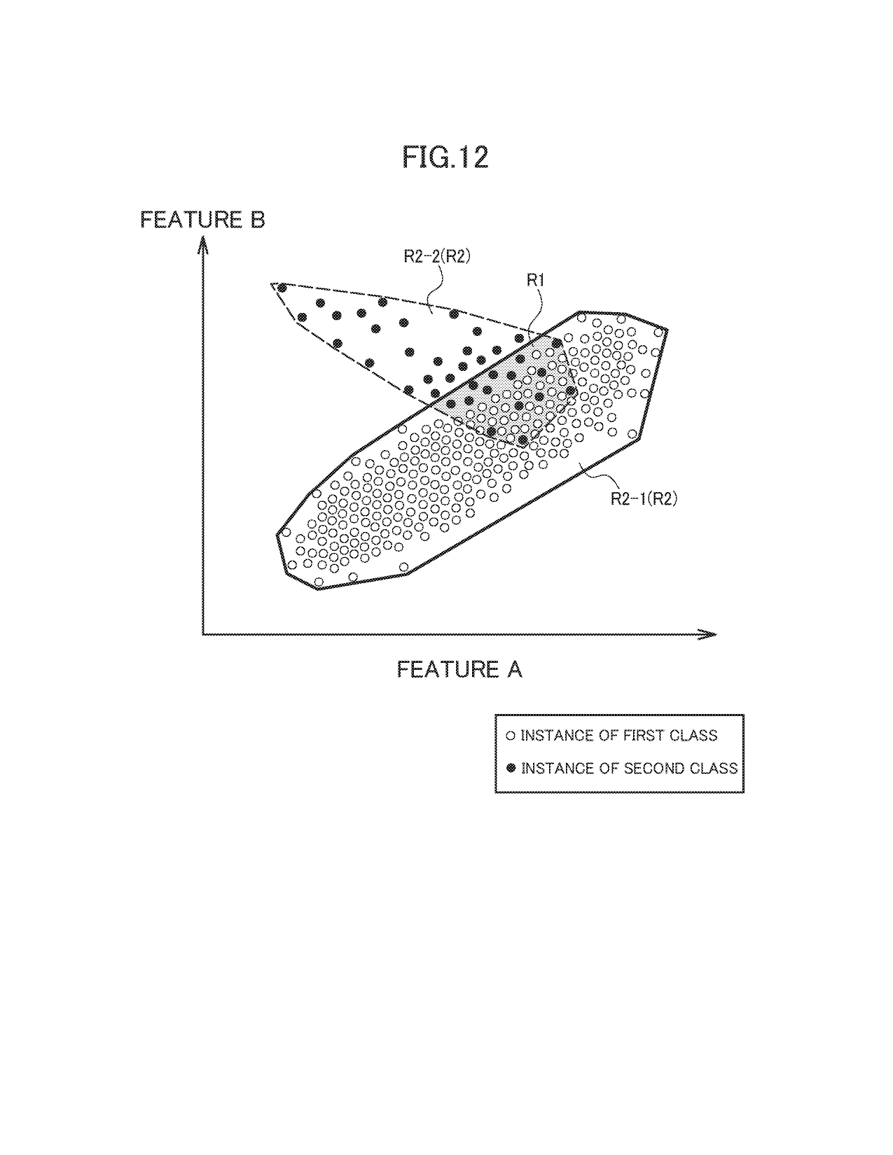

In the following, as illustrated in FIG. 12, an intersection of the set regions is referred to as first region R1, and a region excluding the first region R1 from a union of the set regions is referred to as second region R2. If necessary, a region of the second region R2 that includes a set of instances belonging to the first class is distinguished as "first second region R2-1", and a region of the second region R2 that includes a set of instances belonging to the second class is distinguished as "second second region R2-2". If there is no need to distinguish between "first second region R2-1" and "second second region R2-2", they are collectively referred to simply as "second region R2". It is not easy to determine the overlap between the first and second classes based on the set of instances (set of points). However, the first region R1 can be set by using the convex hull as described above, so that the overlap between the first and second classes can be easily grasped.

In the present embodiment, the training data is newly generated in step S100 described later. The training data is generated so that the feature pertaining to the newly generated training data is located in the first region R1 more often than the feature pertaining to the newly generated training data is located in the second region R2. As used herein "the feature pertaining to the newly generated training data is located in the first region R1 more often than the feature pertaining to the newly generated training data is located in the second region R2" may include that there is no case where the feature pertaining to the newly generated training data is located in the second region R2. In other words, in the present embodiment, the number of pieces of training data that are newly generated and located in the second region R2 may be zero, and all of the features pertaining to the newly generated training data may be located in the first region R1, which will be detailed later. Based on the above, the present embodiment relates to the training data creating device 100 including the processor 110 that performs processing of generating training data for machine learning. The processor 110 plots a plurality of endoscopic images of the training device 90 in the feature space based on individual features, determines classes for the endoscopic images, and sets a set region for each of the determined classes. The processor 110 newly generates training data so that the number of pieces of training data newly generated in the first region R1, which is the intersection region of the set regions for each class, is greater than the number of pieces of training data newly generated in the second region R2, which is the region excluding the first region R1 from the union region of the set regions for each class.

In this way, the training data creating device 100 in the present embodiment determines a class based on the endoscopic image, which is training data, and plots a feature based on the endoscopic image in the feature space, so that the set region for each determined class can be set in the feature space. Since more training data is generated in the first region R1 than in the second region R2, more training data based on features belonging to different classes can be generated in a region with similar features. This allows more machine learning to be performed based on endoscopic images with similar features, so that a more accurate classification result can be output when an endoscopic image is input in the inference phase after machine learning. As a result, it is possible to construct the training data creating device 100 that achieves both improvement in the accuracy of machine learning and increase of training data.

For example, when the features of an endoscopic image pertaining to process K and of an endoscopic image pertaining to process (K+1) are similar, there is a possibility that an endoscopic image that should be classified to process K is incorrectly classified to other processes, such as process (K+1), even if inference is performed by inputting the endoscopic image that should be classified to process K, because there is less endoscopic image training data in process K as described above with reference to FIG. 8. Even if additional training is performed by increasing the training data as in a conventional method, the accuracy of classification is not necessarily improved in the inference phase because training data with appropriate features is not necessarily increased. In this regard, by applying the technique of the present embodiment, more training data can be generated such that instances are increased in the first region R1 where features are similar between two classes. This ensures that the accuracy of classification of endoscopic images can be improved by performing inference using the trained model 22 trained by machine learning with such training data.

In the technique described in Japanese Unexamined Patent Application Publication No. 2022-118654, there is a possibility that the accuracy of machine learning is not sufficiently improved due to creation of training data that differs from originally captured image data. Training data may be deleted so that the amount of training is uniform, but the accuracy of machine learning may not be improved depending on a deletion method. Therefore, it is desirable to propose a technique that can achieve both improvement in the accuracy of machine learning and increase of training data.

The technique of the present embodiment may also be implemented as the information support device 10. In other words, the present embodiment relates to the information support device 10 including the memory 20 that stores the trained model 22 trained by machine learning with training data generated by the training data creating device 100 described above, and the processor 30. The processor 30 outputs a result inferred from the endoscopic image based on the trained model 22. In this way, effects similar to those described above can be achieved.

The technique of the present embodiment may also be implemented as the endoscope system 1. In other words, the endoscope system 1 in the present embodiment includes the information support device 10 described above and the endoscope 3. In this way, effects similar to those described above can be achieved.

The technique of the present embodiment may also be implemented as a training data creating method. In other words, the training data creating method in the present embodiment allows a computer to perform the steps of: plotting a plurality of endoscopic images of the training device 90 in a feature space based on individual features; determining classes for the endoscopic images; and setting a set region for each of the determined classes. The training data creating method allows the computer to further perform the step of newly generating training data so that the number of pieces of training data newly generated in the first region R1, which is the intersection region of the set regions for each class, is greater than the number of pieces of training data newly generated in the second region R2, which is the region excluding the first region R1 from the union region of the set regions for each class. In this way, effects similar to those described above can be achieved.

The technique of the present embodiment may be implemented as a non-transitory information storage medium that stores a program therein. In other words, the non-transitory information storage medium in the present embodiment stores a program that causes a computer to perform the steps of: plotting a plurality of endoscopic images of the training device 90 in a feature space based on individual features; determining classes for the endoscopic images; and setting a set region for each of the determined classes. The non-transitory information storage medium in the present embodiment further stores a program that causes a computer to perform the step of newly generating training data so that the number of pieces of training data newly generated in the first region R1, which is the intersection region of the set regions for each class, is greater than the number of pieces of training data newly generated in the second region R2, which is the region excluding the first region R1 from the union region of the set regions for each class. In this way, effects similar to those described above can be achieved.

In the training data creating device 100 in the present embodiment, the processor 110 may set the set region for each class by forming a convex hull. In this way, the first region R1, which is the overlapping region of the set regions of respective classes in the feature space, can be easily set.

In the training data creating device 100 in the present embodiment, the processor 110 may generate training data in the set region of the second class in the first region R1 where the set region of the first class overlaps the set region of the second class which has less training data than the first class. In this way, more training data pertaining to features belonging to the second class can be generated in the first region R1, which is a region with similar features. As a result, when an endoscopic image to be classified into the second class is input as input data in the inference phase, the trained model 22 can improve the accuracy of classifying the endoscopic image into the second class as output data.

In the training data creating device 100 in the present embodiment, the processor 110 may set a set region based on the first distance, which is the distance in the feature space from the centroid of the features of a plurality of endoscopic images belonging to the same class to the feature of each of the endoscopic images. In this way, the set region can be set appropriately.

In the training data creating device 100 in the present embodiment, the processor 110 may further set the second distance for each class, compare the first distance with the second distance for each instance of the endoscopic image, and set a set region for a set of instances with the first distance shorter than the second distance. In this way, the set region can be set more appropriately.

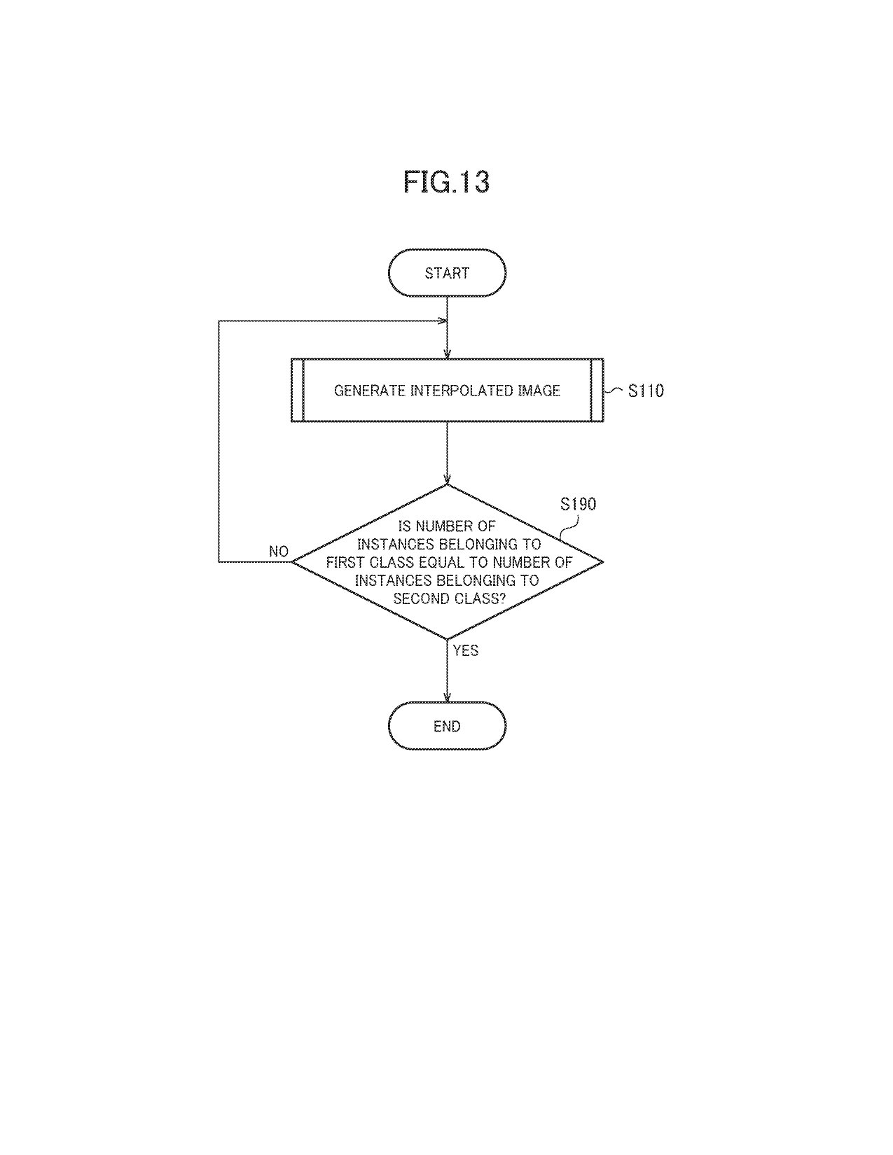

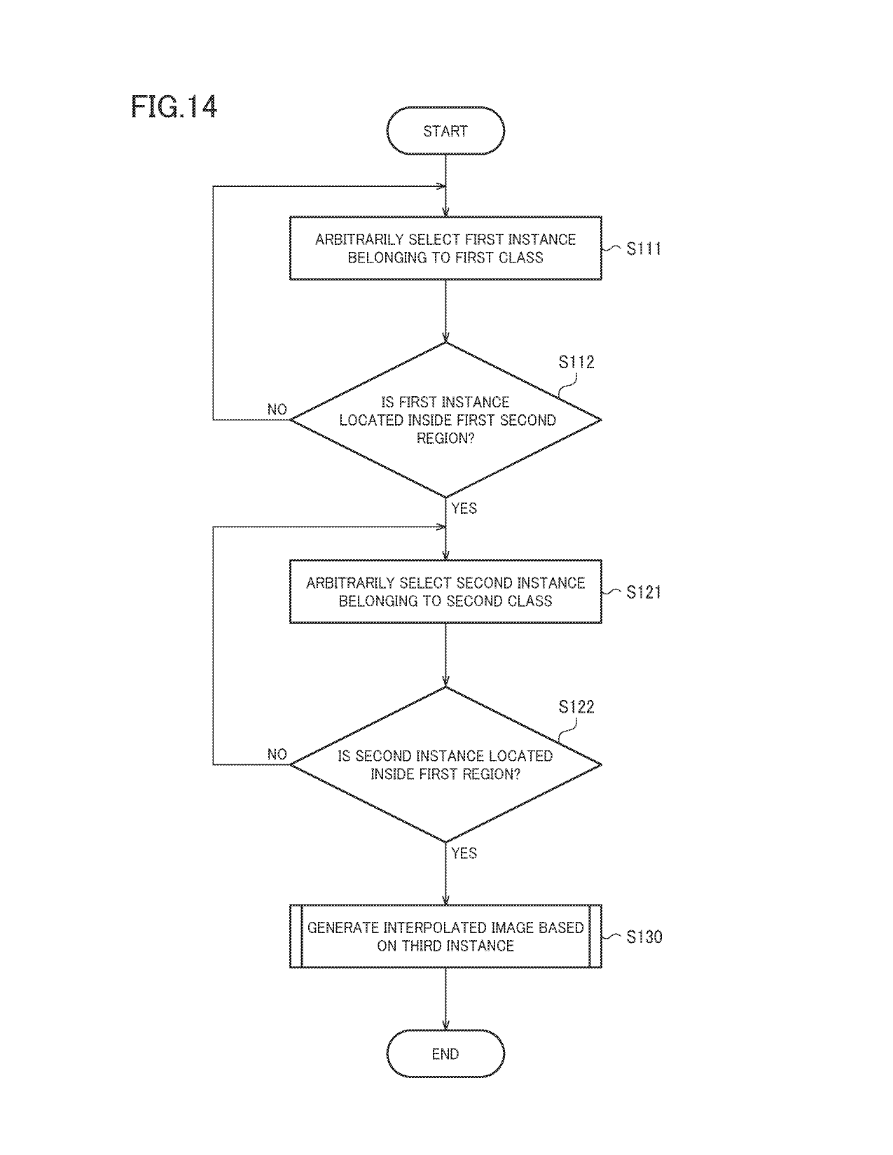

FIG. 13 is a flowchart illustrating step S100 in FIG. 9 in more detail. In FIG. 13, the processor 110 generates an interpolated image (step S110) and determines whether the number of instances belonging to the first class is equal to the number of instances belonging to the second class (step S190). Step S110 will be detailed later with reference to FIG. 14. If the number of instances belonging to the first class is equal to the number of instances belonging to the second class (YES in step S190), the processor 110 terminates the flow. On the other hand, if the number of instances belonging to the first class is different from the number of instances belonging to the second class (NO in step S190), the processor 110 performs step S110 again. In other words, step S110 is processing that increases the number of instances belonging to the second class, as described later, and step S110 is repeatedly performed until the number of instances belonging to the second class becomes equal to the number of instances belonging to the first class.

The criterion in step S190 is that the number of instances belonging to the first class is equal to the number of instances belonging to the second class, but is not limited to this. For example, if the number of instances belonging to the second class is within a predetermined ratio relative to the number of instances belonging to the first class, the processor 110 may determine YES in step S190. The predetermined ratio may be determined as appropriate according to the accuracy of inference obtained as a result of performing training.

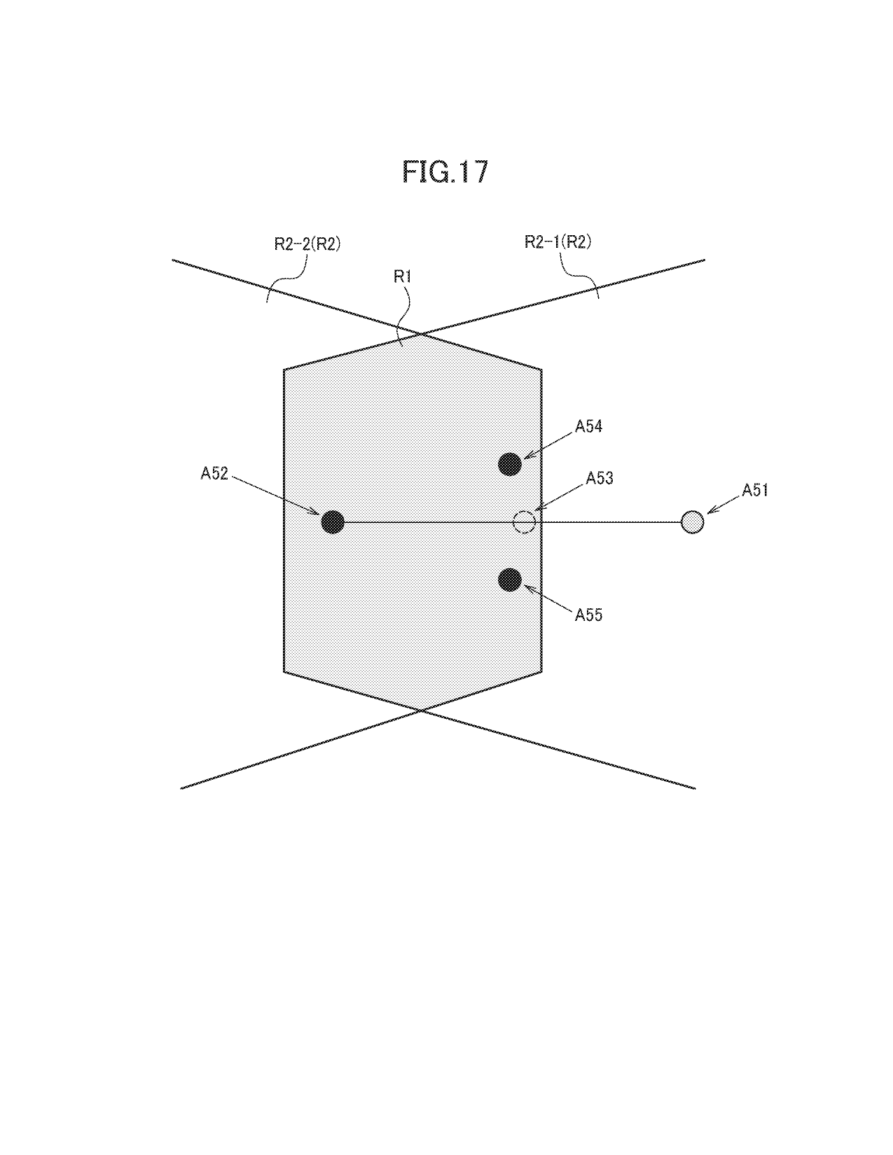

Step S110 will be described in more detail using the flowchart in FIG. 14. The processor 110 arbitrarily selects an instance belonging to the first class (step S111). In the following, the instance belonging to the first class selected by the processing in step S111 is referred to as the first instance. The processor 110 determines whether the first instance is located in the first second region R2-1 (step S112). If the first instance is not located in the first second region R2-1 (NO in step S112), the processor 110 performs step S111 again. On the other hand, if the first instance is located in the first second region R2-1 (YES in step S112), the processor 110 arbitrarily selects an instance belonging to the second class (step S121). In the following, the instance belonging to the second class selected by the processing in step S112 is referred to as the second instance. The first and second instances serve to determine the position of a third instance, as described later with reference to FIG. 17.

The processor 110 then determines whether the second instance is located in the first region R1 (step S122). If the second instance is not located in the first region R1 (NO in step S122), the processor 110 performs step S121 again. On the other hand, if the second instance is located in the first region R1 (YES in step S122), the processor 110 generates an interpolated image based on the third instance (step S130).

In this way, in the training data creating device 100 in the present embodiment, the set regions for each class include the set region of the first class and the set region of the second class. Further, the processor 110 arbitrarily selects the first instance from the set region of the first class and arbitrarily selects the second instance from the set region of the second class. Further, the processor 110 generates training data if the selected first instance is included in the first second region R2-1, which is the set region excluding the first region R1 from the set region of the first class, and the selected second instance is included in the first region R1. In this way, the positions of the first and second instances can be set appropriately. As a result, the position of the instance (the third instance described later) pertaining to a suitable interpolated image can be determined.

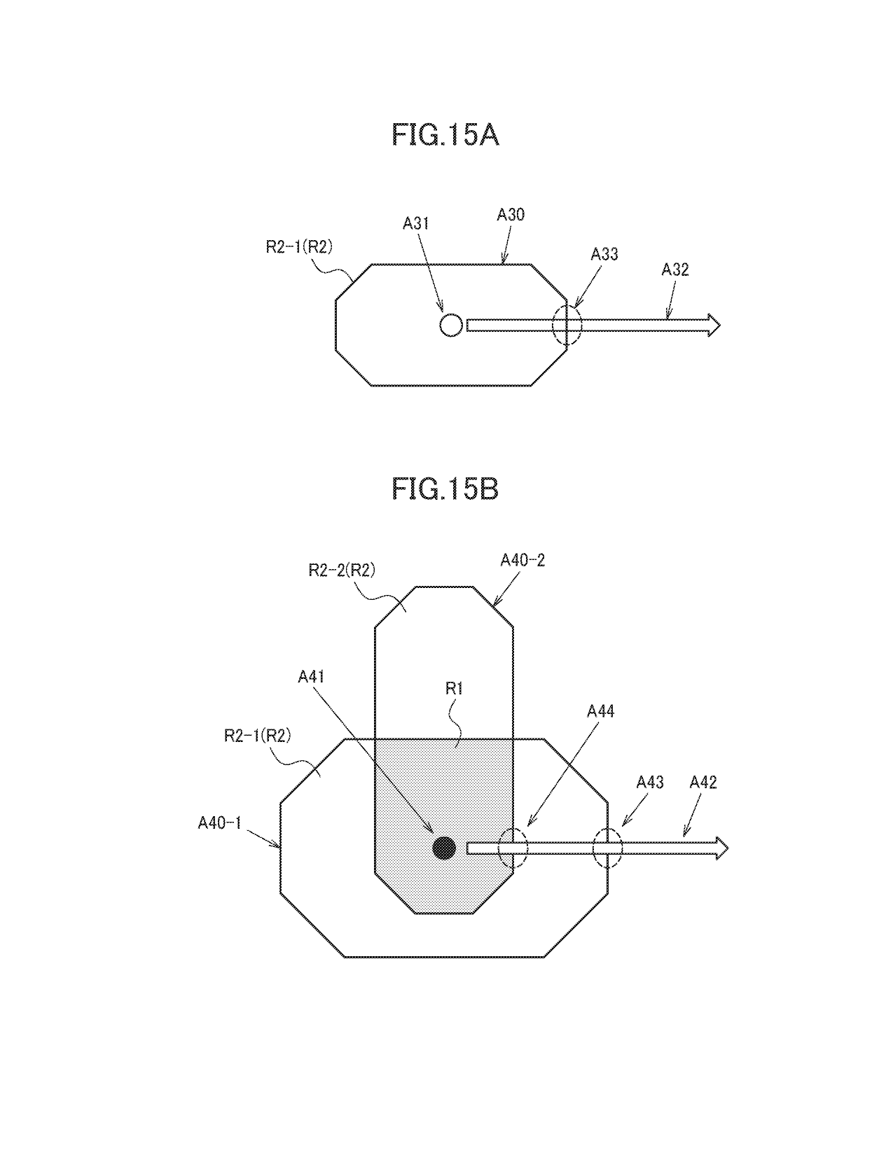

In step S112, whether the first instance is located in the first second region R2-1 can be determined, for example, using the following technique. For example, in FIG. 15A, it is assumed that a convex hull denoted by A30 is the set region of the first class, and the first instance selected in step S111 is located at a position denoted by A31. The processor 110 then finds a search vector denoted by A32. The search vector is a vector for searching for the outer peripheral line of the set region of the first class, with the first instance denoted by A31 as a starting point. In FIG. 15A, the direction of the search vector is parallel to a right direction on the paper, but it may be a left direction, an upward direction, or a downward direction. In other words, the direction of the search vector may be parallel to either the horizontal direction or the vertical direction. The search vector denoted by A32 can also be regarded as a half-line with the first instance denoted by A31 as an endpoint.

Alternatively, one axis of the feature space (hereinafter referred to as "first axis" for convenience) may be arbitrarily selected, and the search vector may be set in an orientation parallel to the selected first axis. In FIG. 15A, it is assumed that the horizontal direction on the paper is parallel to a direction along the selected first axis in

the feature space illustrated in FIG. 10 and the like, and the vertical direction on the paper is parallel to a direction along a second axis selected arbitrarily among the axes perpendicular to the first axis. This is applicable to FIG. 15B described later.

The processor 110 then determines how many times the search vector denoted by A32 in FIG. 15A intersects the outer peripheral line of the set region of the first class. Specifically, since the outer peripheral line of the set region of the first class and the search vector intersect once at a location denoted by A33, it can be determined that the first instance denoted by A31 is located inside the set region of the first class. In other words, the processor 110 determines that the first instance is not located inside the set region of the first class if the outer peripheral line of the set region of the first class and the search vector do not intersect at all or if the outer peripheral line of the set region of the first class and the search vector intersect two or more times.

In step S112, the processor 110 also determines how many times the search vector denoted by A32 intersects the outer peripheral line of the set region of the second class. Although not illustrated in FIG. 15A, the processor 110 determines that the first instance is not located inside the set region of the second class if the outer peripheral line of the set region of the second class and the search vector do not intersect at all or if the outer peripheral line of the set region of the first class and the search vector intersect two or more times. Then, if the first instance is located inside the set region of the first class and not located inside the set region of the second class, the processor 110 determines YES in step S112 because the first instance is located in the first second region R2-1.

In step S122, the technique described above in step S112 can also be used as to whether the second instance is located in the first region R1. For example, in FIG. 15B, it is assumed that the convex hull denoted by A40-1 is the set region of the first class, the convex hull denoted by A40-2 is the set region of the second class, and the second instance selected in step S121 is located at the position denoted by A41. The processor 110 then finds a search vector denoted by A42. It can be determined that the first instance denoted by A41 is located inside the first region R1, because the outer peripheral line of the set region of the first class and the search vector intersect once at the location denoted by A43, and the outer peripheral line of the set region of the second class and the search vector intersect once at the location denoted by A44.

As in the case described above with reference to FIG. 15A, the search vector denoted by A42 in FIG. 15B can also be considered as a half-line with the second instance denoted by A41 as an endpoint. Based on the above, in the training data creating device 100 in the present embodiment, the processor 110 virtually sets a half-line parallel to one axis arbitrarily selected in the feature space and with the selected second instance as an endpoint. If the set half-line intersects the outer peripheral line of the set region of the first class once and intersects the outer peripheral line of the set region of the second class once, the processor 110 determines that the second instance is present inside the first region R1 and generates training data. In this way, it can be easily determined whether the first and second instances are located within a suitable region in the feature space.

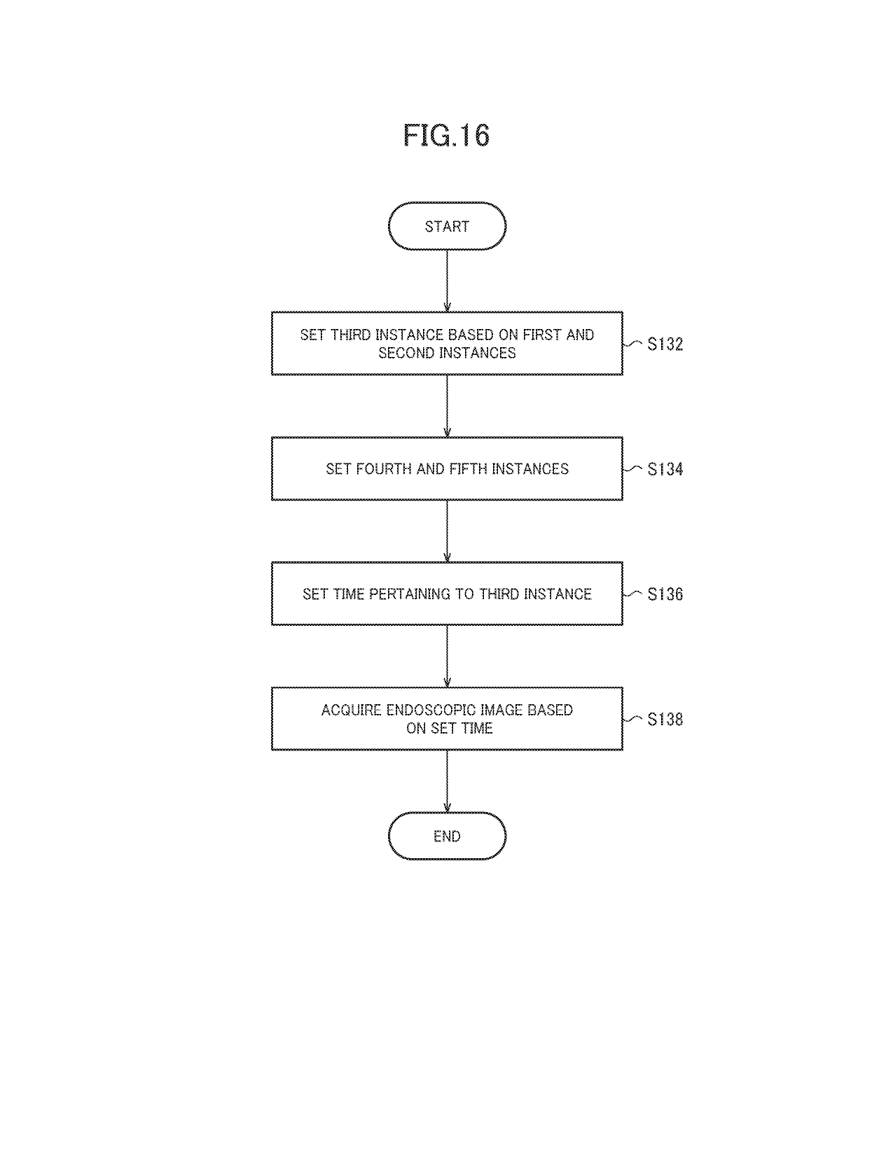

Step S130 will be described in more detail using the flowchart in FIG. 16. The processor 110 sets a third instance based on the first and second instances (step S132) and then sets fourth and fifth instances (step S134). The processor 110 then sets the time pertaining to the third instance (step S136) and acquires an endoscopic image based on the determined time (step S138).

For example, as illustrated in FIG. 17, since a part of the convex hull of the set region of the first class and a part of the convex hull of the set region of the second class overlap, the first region R1, the first second region R2-1, and the second second region R2-2 are defined. It is assumed that the first instance denoted by A51 is selected in step S111 in FIG. 14. In this case, the processor 110 determines YES in step S112 in FIG. 14 because the first instance denoted by A51 in FIG. 17 is located inside the first second region R2-1. Similarly, it is assumed that the second instance denoted by A52 in FIG. 17 is selected in step S121 in FIG. 14. In this case, the processor 110 determines YES in step S122 in FIG. 14 because the second instance denoted by A52 is located inside the first region R1.

The processor 110 then sets the third instance at a position denoted by A53 in

FIG. 17 in step S132. Step S132 is processing of determining the position of a feature newly interpolated. The position denoted by A53 is set to be a position on a line segment connecting the position of the first instance denoted by A51 and the position of the second instance denoted by A52 and inside the first region R1. For example, the processor 110 may perform control such that the probability of setting the third instance in the first region R1 is higher than the probability of setting the third instance in the first second region R2-1. The position denoted by A53 is set near the boundary line between the first region R1 and the first second region R2-1 and inside the first region R1. In other words, even if the feature space is a nonlinear space, the third instance is set so that locally the feature space can be treated as a linear space. This can make the set region of the second class clearer. To determine whether linearity is satisfied within a desired range of the feature space, for example, a plurality of instances may be selected at random within the desired range, and whether additivity and homogeneity are satisfied may be checked for the selected instances.

The processor 110 then sets the fourth instance denoted by A54 and the fifth instance denoted by A55 in FIG. 17 in step S134 in FIG. 16. The fourth instance denoted by A54 and the fifth instance denoted by A55 are instances based on the feature closest to the position denoted by A53 among the instances belonging to the second class. As described above, since the endoscopic images in the present embodiment are images acquired regularly every first time based on the endoscopic video, the time when the endoscopic image based on the fourth instance denoted by A54 is acquired and the time when the endoscopic image based on the fifth instance denoted by A55 is acquired are known. Similarly, the time when the endoscopic image based on the second instance denoted by A52 is acquired is also known.

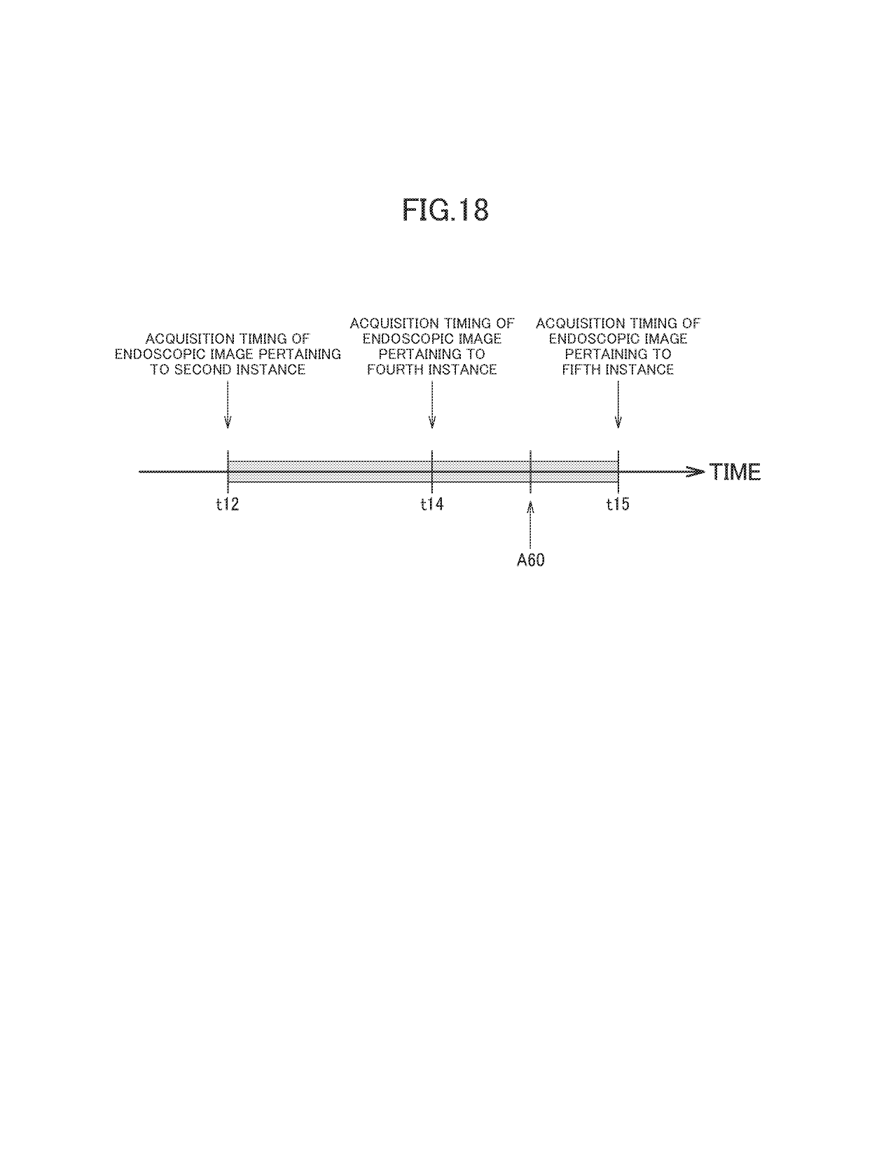

Step S136 will be described in more detail. For example, as illustrated in FIG. 18, it is assumed that the endoscopic image pertaining to the second instance is acquired from the endoscopic video at timing t12, the endoscopic image pertaining to the fourth instance is acquired from the endoscopic video at timing t14, which is later than timing t12, and the endoscopic image pertaining to the fifth instance is acquired from the endoscopic video at timing t15, which is later than timing t14. FIG. 18 is an example and is not intended to limit the temporal order of timing t12, timing t14, and timing t15.

In step S136, for example, the processor 110 generates time information according to random numbers that are uniform over a time range from timing t12 to timing t15, with a second time as the smallest unit. The calculation of uniform random numbers can be performed using a known method. The second time is preferably shorter than the first time described above. More specifically, for example, when the endoscopic images are acquired every 2 seconds (= first time), in step S136, the processor 110 generates time information according to random numbers that are uniform over a time range from timing t12 to timing t15, with 1 second (= second time) as the smallest unit. As a result, for example, the time denoted by A60 in FIG. 18 is determined as the time based on the newly created third instance. In this way, training data can be newly created which is considered reasonable as an endoscopic image pertaining to the interpolated feature in the feature space.

Then, in step S138 in FIG. 16, the processor 110 acquires the endoscopic image based on the time denoted by A60 in FIG. 18. As a result, the training data based on the third instance is interpolated.

In step S136, the processor 110 may generate time information according to random numbers that are uniform over a time range from timing t14 to timing t15, with the second time as the smallest unit. This is because the third instance is located nearest to the fourth and fifth instances in the feature space, and therefore the time pertaining to the third instance is assumed to be close to the fourth and fifth instances.

When the third instance is set inside the first second region R2-1, the fourth and fifth instances may be set as instances based on the feature closest to the position of the set third instance among the instances belonging to the first class. The processor 110 then may acquire the time pertaining to the third instance based on the times pertaining to the first, fourth, and fifth instances. Based on the above, in the training data creating device 100 in the present embodiment, the processor 110 interpolates a new feature inside the first region R1 based on the acquisition times of the endoscopic images pertaining to a plurality of instances and thereby creates training data corresponding to the interpolated feature. In this way, the acquisition time pertaining to the endoscopic image pertaining to the newly interpolated feature can be estimated within a reasonable range. As a result, an endoscopic image can be newly created as appropriate training data.

In this way, by applying the technique of the present embodiment, it is possible to newly create training data based on the instance belonging to the second class. In the sigmoidectomy described above, for example, process 3 (vascular treatment) in FIG. 5 requires a shorter time than the process pertaining to medial mobilization, which is preparation and post-treatment of treating blood vessels, such as processes 2 and 4, and thus less training data is acquired through endoscopic surgery. It is conceivable to apply the technique of the present embodiment with process 3 as the second class. In other words, in the training data creating device 100 in the present embodiment, the processor 110 arbitrarily selects the second instance from the set region of the second class, which is a class related to the process of treating blood vessels in sigmoidectomy. In this way, the trained model 22 can improve the accuracy of classifying the process of treating blood vessels and outputting the classified process as output data when an endoscopic image is input as input data in sigmoidectomy

Further, for example, since process 7 (rectal dissection) in FIG. 5 requires a shorter time than process 6 (mesorectal treatment) and process 8 (rectal anastomosis), which are preparation and post-treatment for dissection, less training data is acquired through endoscopic surgery. It is conceivable to apply the technique of the present embodiment with process 7 as the second class. In other words, in the training data creating device 100 in the present embodiment, the processor 110 arbitrarily selects the second instance from the set region of the second class, which is a class related to the process of dissecting the rectum in sigmoidectomy. In this way, when an endoscopic image is input as input data in sigmoidectomy, the accuracy of classifying the process of dissecting the rectum and outputting the classified process as output data can be improved.

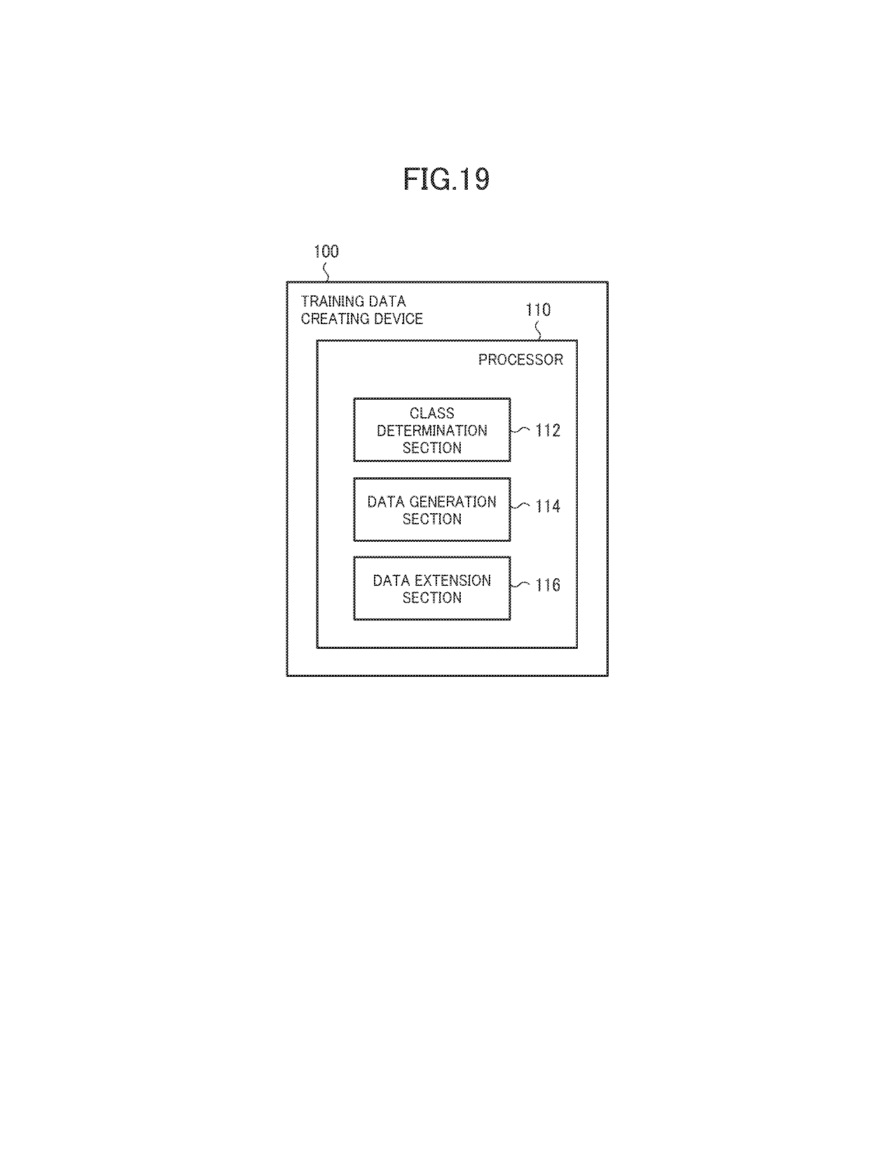

The training data creating device 100 in the present embodiment may be, for example, a configuration example illustrated in FIG. 19. The training data creating device 100 illustrated in FIG. 19 differs from the training data creating device 100 illustrated in FIG. 1 in that it further includes a data extension section 116. In other words, the training data creating device 100 in the present embodiment may further store a program that allows the processor 110 to function as the data extension section 116 in a not-illustrated memory or the like.

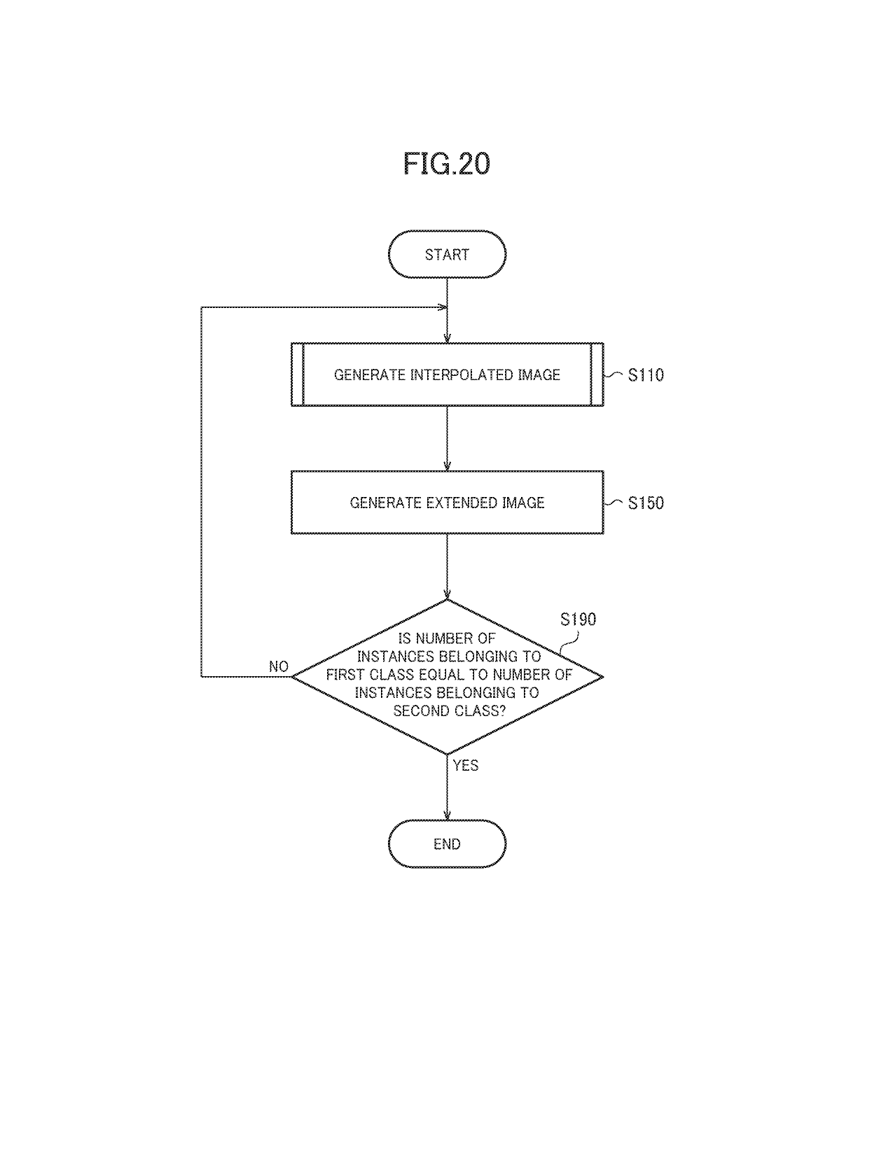

When the training data creating device 100 illustrated in FIG. 19 is used, the flowchart illustrated in FIG. 13 may be modified to the flowchart illustrated in FIG. 20. The flowchart in FIG. 20 differs from the flowchart in FIG. 13 in that processing of the processor 110 generating an extended image (step S150) is added between steps S110 and S190 described above.

More specifically, for example, step S150 performs processing such as scaling, shearing, horizontal flipping, vertical flipping, and rotation on the endoscopic image acquired in step S10 in FIG. 9. Scaling here refers to enlarging or reducing the endoscopic image within a specified range. Shearing refers to transforming a rectangular endoscopic image into a parallelogram. Horizontal flipping refers to reversing the endoscopic image with respect to a straight line passing through the center of the endoscopic image and parallel to the vertical direction of the endoscopic image. Vertical flipping refers to reversing the endoscopic image with respect to a straight line passing through the center of the endoscopic image and parallel to the horizontal direction of the endoscopic image. When the technique of the present embodiment is applied to sigmoidectomy, the processing of flipping the endoscopic image upside down does not have to be included in step S150. This is because the possibility of acquiring an image reversed upside down is low in sigmoidectomy using a rigid endoscope.

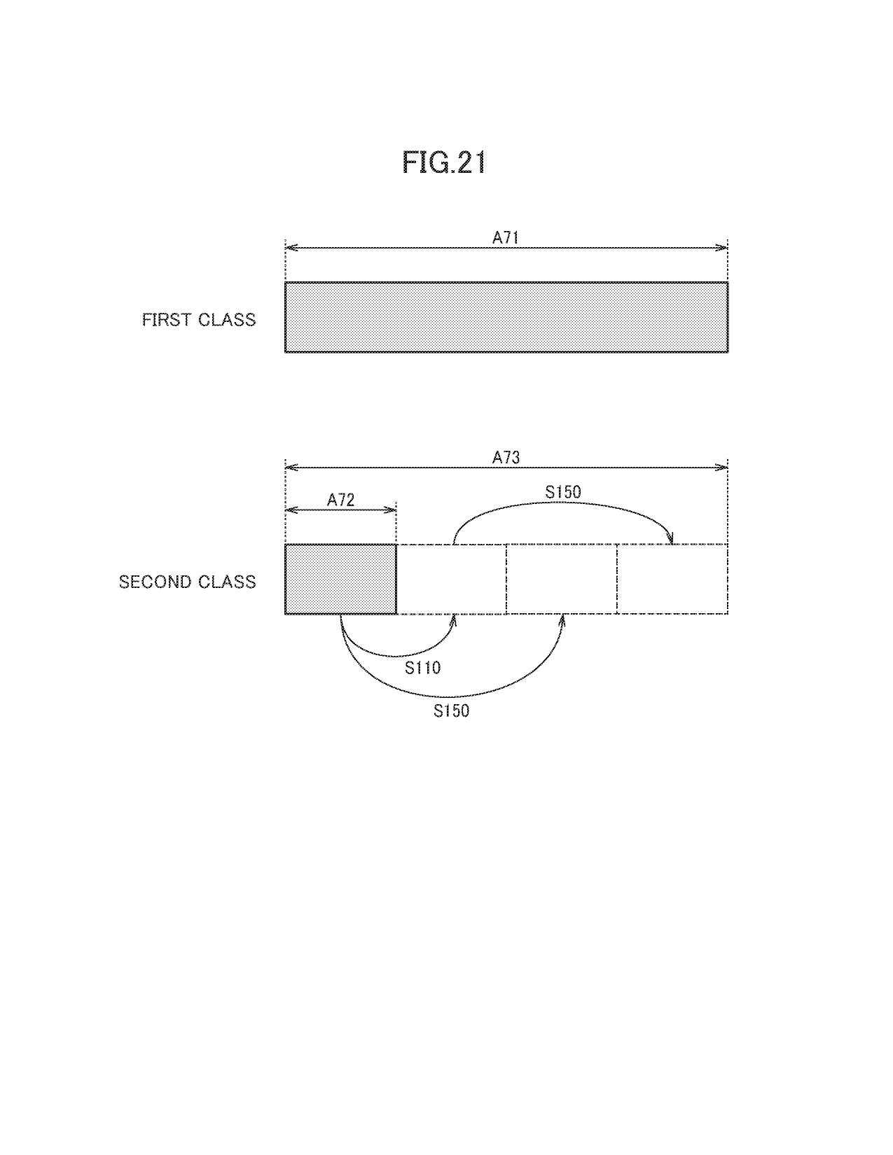

Step S150 in FIG. 20 may be further applied to the interpolated image generated in step S110. For example, it is assumed that when step S10 in FIG. 9 is performed, the number of endoscopic images belonging to the first class corresponds to a length denoted by A71 in FIG. 21, and the number of endoscopic images belonging to the second class corresponds to a length denoted by A72. In this case, the processor 110 newly creates an interpolated image newly created in step S110 in FIG. 20, a deformed image newly created in step S150, and an image in which the interpolated image further undergoes the processing in step S150, for the endoscopic image belonging to the second class acquired in step S10 in FIG. 9. This increases the number of endoscopic images belonging to the second class to the number corresponding to a length denoted by A73 in FIG. 21. When the length denoted by A73 becomes equal to the length denoted by A71, the processor 110 determines YES in step S190 described above.

Based on the above, in the training data creating device 100 in the present embodiment, the processor 110 further generates training data by transforming a plurality of endoscopic images of the training device 90 and the endoscopic image pertaining to the generated training data. In this way, the number of pieces of training data pertaining to the endoscopic images belonging to the second class can be increased.

In the example described above, as long as the number of pieces of training data for the process pertaining to the first class is greater than the number of pieces of training data for the process pertaining to the second class, there are no restrictions on other relationships. However, in addition to the relationship described above, for example, when the process pertaining to the first class and the process pertaining to the second class have a transitional relationship, the technique of the present embodiment may be modified and implemented as follows. More specifically, although the flowchart is not illustrated, step S110 may be modified and implemented as follows.

In the present embodiment, transition of processes refers to the processes proceeding according to a predetermined endoscopic surgical plan, but also includes the transition between processes that is feasible as treatment. For example, in principle, the treatment proceeds according to process numbers, but processes may proceed in the reverse order of the process numbers for a given reason. Even for the processes that proceed in this case, it can be said that the processes transition. The process may return to the previous step or may return two or more steps. The given reason is, for example, that the treatment of a treatment target is difficult, or that the treatment in a process is insufficient. For example, suppose a given endoscopic surgery that includes process M, process (M+1), process (M+2), and process (M+3). In principle, the processes proceed in the order of process M, process (M+1), and process (M+2). However, if treatment is performed from process (M+2) back to process M due to insufficient treatment in process M or for other reasons, process M and process (M+2) can be said to have a transitional relationship. For example, the processor 110 stores a process transition pattern based on a predetermined plan and a process transition pattern actually performed in a not-illustrated memory, through each endoscopic surgery. The processor 110 then creates new training data from only the processes that have a transitional relationship, based on the stored process transition patterns. In this way, it is possible to prevent the creation of new training data from processes that are in a non-transitional relationship with each other. As a result, it is possible to prevent degradation of the performance of the trained model 22.

For example, the processor 110 arbitrarily selects one instance that is included in the first region R1 and belongs to the second class, and then arbitrarily selects one instance that is included in the first second region R2-1 and belongs to the first class. Specifically, for example, as illustrated in FIG. 22, an instance denoted by A80 is the instance that is included in the first region R1 and belongs to the second class (i.e., second instance), and an instance denoted by A81 is the instance that is included in the first second region R2-1 and belongs to the first class (i.e., first instance). In FIG. 22, the first region R1, the first second region R2-1, and the second second region R2-2 are illustrated in the same manner as in FIG. 17.

The processor 110 then connects the instance denoted by A80 (i.e., second instance) and the instance denoted by A81 (i.e., first instance) with a virtual line segment. The processor 110 then sets the third instance within a set of points pertaining to a line segment indicated by a solid line in A82 in the connecting line segment. In other words, in the example illustrated in FIG. 22, the processor 110 does not set the third instance within a set of points indicated by a dotted line A83. In other words, if the process pertaining to the first class and the process pertaining to the second class have a transitional relationship, the processor 110 generates new training data so that the feature is located in the first region R1, but does not generate new training data so that the feature

is located in the first second region R2-1. This is because if the process pertaining to the first class and the process pertaining to the second class are transitional processes, training data pertaining to the second class should be newly generated. In the case of the example in FIG. 22, the position of the third instance to be newly set is not required to be near the boundary line between the first region R1 and the first second region R2-1 as long as it is on the solid line portion denoted by A82. After the third instance is set, the fourth and fifth instances are set, the time pertaining to the third instance is set, and the endoscopic image based on the set time is acquired, in the same manner as described above.

For example, a new instance may be set continuously on the solid line portion denoted by A82 in FIG. 22. Specifically, for example, the processor 110 sets the third instance near the boundary line between the first region R1 and the first second region R2-1 in FIG. 22, and sets the time pertaining to the third instance. The processor 110 then obtains the difference between the time pertaining to the set third instance and the time of the other instance, and sets a new sixth instance pertaining to the time in consideration of the difference to the time pertaining to the third instance, at a desired position on the solid line portion denoted by A82. The other instance can be selected as appropriate from the second instance denoted by A80, the fourth instance, and the fifth instance. By repeating the same technique, three or more instances may be newly set on the solid line denoted by A82, in addition to the third and sixth instances described above.

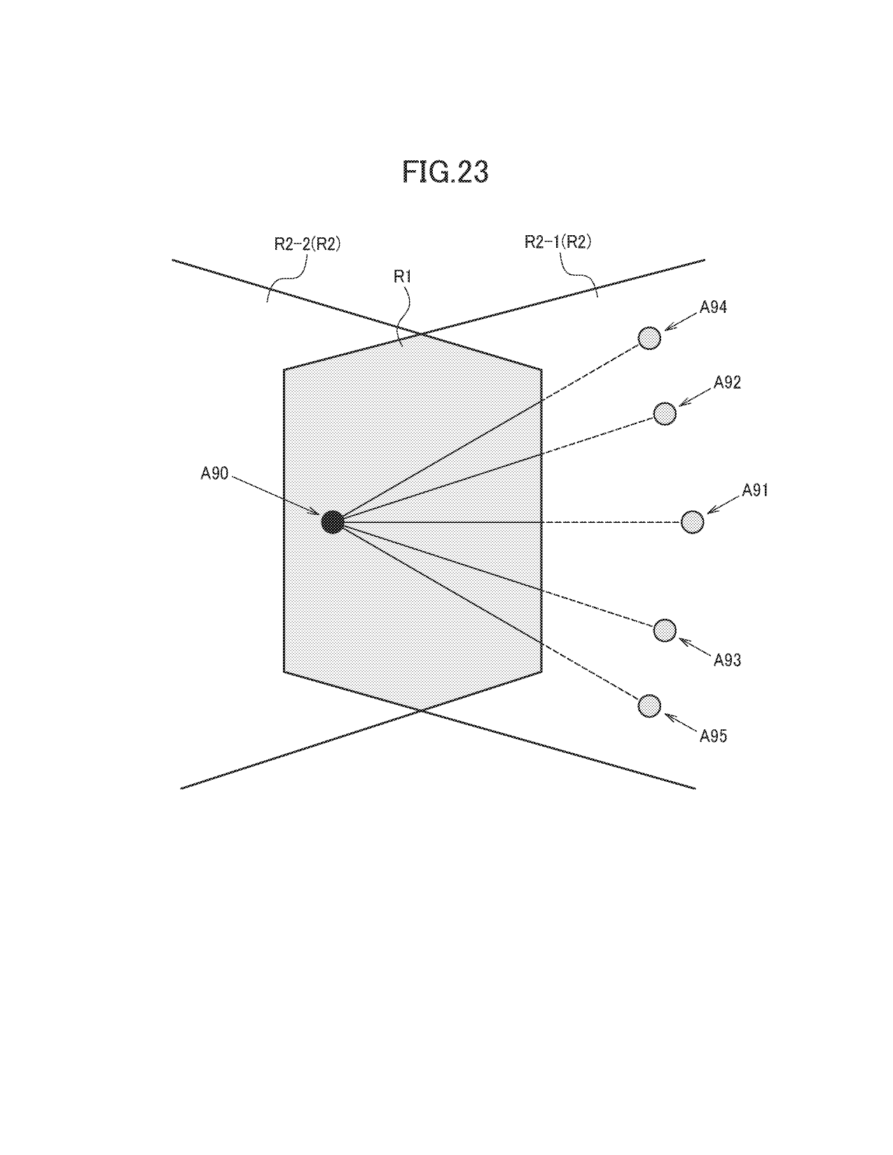

When training data is repeatedly generated by the technique illustrated in FIG. 22, one instance that is newly included in the first second region R2-1 and belongs to the first class may be selected arbitrarily, without selecting again one instance that belongs to the second class. In other words, for example, when five pieces of training data are to be newly generated, the processor 110 may select an instance denoted by A90 in FIG. 23 and select instances denoted by A91, A92, A93, A94, and A95. The instance denoted by A90 is the instance that is included in the first region R1 and belongs to the second class, and the instances denoted by A91 to A95 are the instances that are included in the first second region R2-1 and belong to the first class.

The processor 110 then, for example, connects the instance denoted by A90 and the instance denoted by A91 in FIG. 23 with a line segment, sets the third instance for a point on a solid line portion of the connecting line segment, then sets the fourth and fifth instances, sets the time pertaining to the third instance, and acquires an endoscopic image based on the set time, in the same manner as described above. The processor 110 may repeatedly perform similar processing for the instances denoted by A90 and A92, the instances denoted by A90 and A93, the instances denoted by A90 and A94, and the instances denoted by A90 and A95.

As described above with reference to FIG. 22, the processor 110 may continuously set a plurality of instances on the solid line portion of the line segment connecting the instance denoted by A90 and the instance denoted by A91 in FIG. 23. Similarly, the processor 110 may continuously set a plurality of instances on the solid line portion of the line segment connecting the instance denoted by A90 and the instance denoted by A92, or may continuously set a plurality of instances on the solid line portion of the line segment connecting the instance denoted by A90 and the instance denoted by A93. Similarly, the processor 110 may continuously set a plurality of instances on the solid line portion of the line segment connecting the instance denoted by A90 and the instance denoted by A94, or may continuously set a plurality of instances on the solid line portion of the line segment connecting the instance denoted by A90 and the instance denoted by A95.

Based on the above, in the training data creating device 100 in the present embodiment, the processor 110 sets a line segment where a straight line connecting the first instance and the second instance overlaps with the first region R1 in the feature space, and generates training data corresponding to an instance located on the set line segment. In this way, training data based on the instance located inside the first region R1 can be newly created. This increases the training data pertaining to the instance belonging to the second class.

The process pertaining to the first class and the process pertaining to the second class may be transitional processes in the manipulation using the endoscope 3. In this

way, it is possible to construct a technique that increases the training data belonging to the second class pertaining to the process transitional to the process pertaining to the first class. As a result, when an endoscopic image with similar features between the first class and the second class is input as input data to the trained model 22, a more accurate result of classification of the first class and the second class can be output as output data. This allows the user to execute the treatment without confusing the process stages.

The processor 110 may select one second instance and a plurality of first instances, set a plurality of line segments based on the one second instance and the respective first instances, and generate training data on the set line segments. In this way, a plurality of pieces of training data can be newly created without performing processing of selecting the second instance again. As a result, it is possible to create more training data while reducing the processing burden on the training data creating device 100.

Although the embodiments have been described in detail above, it will be readily understood by those skilled in the art that many modifications can be made without substantially departing from the novel matters and effects in the present disclosure. Therefore, all such modifications are intended to be included within the scope of the present disclosure. Any term cited with a different term having a broader meaning or the same meaning at least once in the specification and the drawings can be replaced by the different term in any place in the specification and the drawings. All combinations of the embodiments and modifications are also included in the scope of the present disclosure. The configuration, operation, and the like of the training data creating device, the information support device, the endoscope system, the training data creating method, and the information storage medium are not limited to those described in the present embodiment and can be modified in various ways.

1. A training data creating device comprising one or more processors configured to perform processing of generating training data for machine learning, wherein
the one or more processors
plot a plurality of endoscopic images of a training device in a feature space based on individual features,
determine classes for the endoscopic images,
set a set region for each of the determined classes, and
newly generate the training data so that a number of pieces of the training data newly generated in a first region is greater than a number of pieces of the training data newly generated in a second region, the first region being an intersection region of the set regions for each class, the second region being a region excluding the first region from a union region of the set regions for each class.

2. The training data creating device according to claim 1, wherein
the one or more processors set the set region based on a first distance, the first distance being a distance in the feature space from a centroid of the features of the endoscopic images belonging to a same class to the feature of each of the endoscopic images.

3. The training data creating device according to claim 2, wherein
the one or more processors
further set a second distance for each of the classes,
compare the first distance with the second distance for each of instances of the endoscopic images, and
set the set region for a set of the instances with the first distance shorter than the second distance.

4. The training data creating device according to claim 1, wherein
the one or more processors set the set region for each class by forming a convex hull.

5. The training data creating device according to claim 1, wherein
the one or more processors interpolate a new feature inside the first region, based on an acquisition time of the endoscopic images pertaining to a plurality of instances, and generate the training data corresponding to the interpolated feature.

6. The training data creating device according to claim 1, wherein
the one or more processors generate the training data in the set region of a second class in the first region where the set region of a first class overlaps the set region of the second class having less training data than the first class.

7. The training data creating device according to claim 6, wherein
the set region for each class includes the set region of the first class and the set region of the second class, and
the one or more processors arbitrarily select a first instance from the set region of the first class and a second instance from the set region of the second class, and generate the training data when the selected first instance is included in a first second region and the selected second instance is included in the first region, the first second region being the set region excluding the first region from the set region of the first class.

8. The training data creating device according to claim 7, wherein
the one or more processors virtually set a half-line parallel to one axis arbitrarily selected in the feature space and having the selected second instance as an endpoint, and
when the set half-line intersects an outer periphery of the set region of the first class once and intersects an outer periphery of the set region of the second class once, the one or more processors determine that the second instance is present inside the first region, and generate the training data.

9. The training data creating device according to claim 7, wherein
the one or more processors arbitrarily select the second instance from the set region of the second class, the second class being the class related to a process of treating a blood vessel in sigmoidectomy.

10. The training data creating device according to claim 7, wherein
the one or more processors arbitrarily select the second instance from the set region of the second class, the second class being the class related to a process of dissecting rectum in sigmoidectomy.

11. The training data creating device according to claim 7, wherein
the one or more processors set a line segment where a straight line connecting the first instance and the second instance overlaps with the first region in the feature space, and generate the training data corresponding to an instance located on the set line segment.

12. The training data creating device according to claim 11, wherein
a process pertaining to the first class and a process pertaining to the second class are transitional processes in manipulation using an endoscope.

13. The training data creating device according to claim 12, wherein
the one or more processors
select one the second instance and a plurality of the first instances,
set a plurality of the line segments based on the one second instance and the respective first instances, and
generate the training data on the set line segments.

14. The training data creating device according to claim 1, wherein
the one or more processors further generate the training data by transforming the endoscopic images of the training device and the endoscopic image pertaining to the generated training data.

15. An information support device comprising:
a memory configured to store a trained model trained by machine learning with the training data generated by the training data creating device according to claim 1; and
one or more processors, wherein
the one or more processors output a result of inference from the endoscopic image based on the trained model.

16. An endoscope system comprising:
the information support device according to claim 15; and
an endoscope.

17. A training data creating method comprising:
plotting a plurality of endoscopic images of a training device in a feature space based on individual features;
determining classes for the endoscopic images;
setting a set region for each of the determined classes; and
newly generating training data so that a number of pieces of the training data newly generated in a first region is greater than a number of pieces of the training data newly generated in a second region, the first region being an intersection region of the set regions for each class, the second region being a region excluding the first region from a union region of the set regions for each class.

18. A non-transitory information storage medium that stores a program for causing a computer to execute:
plotting a plurality of endoscopic images of a training device in a feature space based on individual features;
determining classes for the endoscopic images;
setting a set region for each of the determined classes; and
newly generating training data so that a number of pieces of the training data newly generated in a first region is greater than a number of pieces of the training data newly generated in a second region, the first region being an intersection region of the set regions for each class, the second region being a region excluding the first region from a union region of the set regions for each class.
